SEQUENCE LISTING (6 sequences)

SEQ ID NO: 1 - protein, 18 aa, synthetic construct
  REGION 1..18: note = Circular peptide
  VARIANT 1: note = X is a basic amino acid selected from arginine (R), lysine (K), and histidine (H)
  VARIANT 2: note = X is a basic amino acid selected from arginine (R), lysine (K), and histidine (H)
  VARIANT 3: note = X is a basic amino acid selected from arginine (R), lysine (K), and histidine (H)
  VARIANT 4: note = X is a hydrophobic amino acid selected from valine (V), leucine (L), isoleucine (I), and methionine (M)
  VARIANT 6: note = X is an hydrophobic aromatic amino acids selected from phenylalanine (F), tyrosine (Y) and tryptophan (W)
  VARIANT 7: note = X is a hydrophobic amino acid selected from valine (V), leucine (L), isoleucine (I), and methionine (M)
  VARIANT 8: note = X is a basic amino acid selected from arginine (R), lysine (K), and histidine (H)
  SITE 9: note = D-Proline
  VARIANT 10: note = X is a basic amino acid selected from arginine (R), lysine (K), and histidine (H)
  VARIANT 11: note = X is an hydrophobic aromatic amino acids selected from phenylalanine (F), tyrosine (Y) and tryptophan (W)
  VARIANT 12: note = X is a hydrophobic amino acid selected from valine (V), leucine (L), isoleucine (I), and methionine (M)
  VARIANT 13: note = X is a hydrophobic amino acid selected from valine (V), leucine (L), isoleucine (I), and methionine (M)
  VARIANT 15: note = X is a hydrophobic amino acid selected from valine (V), leucine (L), isoleucine (I), and methionine (M)
  VARIANT 16: note = X is a basic amino acid selected from arginine (R), lysine (K), and histidine (H)
  VARIANT 17: note = X is a basic amino acid selected from arginine (R), lysine (K), and histidine (H)
  SITE 18: note = D-Proline
XXXXCXXXPXXXXCXXXP

SEQ ID NO: 2 - protein, 18 aa, synthetic construct
  REGION 1..18: note = Circular peptide
  VARIANT 1: note = X is a basic amino acid selected from arginine (R), lysine (K), and histidine (H)
  VARIANT 2: note = X is a basic amino acid selected from arginine (R), lysine (K), and histidine (H)
  VARIANT 3: note = X is a basic amino acid selected from arginine (R), lysine (K), and histidine (H)
  VARIANT 4: note = X is a hydrophobic amino acid selected from valine (V), leucine (L), isoleucine (I), and methionine (M)
  DISULFID 5..14: note = intrachain disulfide bond
  VARIANT 6: note = X is a hydrophobic aromatic amino acids selected from phenylalanine (F), tyrosine (Y) and tryptophan (W)
  VARIANT 7: note = X is a hydrophobic amino acid selected from valine (V), leucine (L), isoleucine (I), and methionine (M)
  VARIANT 8: note = X is a basic amino acid selected from arginine (R), lysine (K), and histidine (H)
  SITE 9: note = D-Proline
  VARIANT 10: note = X is a basic amino acid selected from arginine (R), lysine (K), and histidine (H)
  VARIANT 11: note = X is a hydrophobic aromatic amino acids selected from phenylalanine (F), tyrosine (Y) and tryptophan (W)
  VARIANT 12: note = X is a hydrophobic amino acid selected from valine (V), leucine (L), isoleucine (I), and methionine (M)
  VARIANT 13: note = X is a hydrophobic amino acid selected from valine (V), leucine (L), isoleucine (I), and methionine (M)
  VARIANT 15: note = X is a hydrophobic amino acid selected from valine (V), leucine (L), isoleucine (I), and methionine (M)
  VARIANT 16: note = X is a basic amino acid selected from arginine (R), lysine (K), and histidine (H)
  VARIANT 17: note = X is a basic amino acid selected from arginine (R), lysine (K), and histidine (H)
  SITE 18: note = D-Proline
XXXXCXXXPXXXXCXXXP

SEQ ID NO: 3 - protein, 18 aa, synthetic construct
  REGION 1..18: note = Circular peptide
  VARIANT 1: note = X is a basic amino acid selected from arginine (R), lysine (K), and histidine (H)
  VARIANT 2: note = X is a basic amino acid selected from arginine (R), lysine (K), and histidine (H)
  VARIANT 3: note = X is a basic amino acid selected from arginine (R), lysine (K), and histidine (H)
  VARIANT 4: note = X is a hydrophobic amino acid selected from valine (V), leucine (L), isoleucine (I), and methionine (M)
  MOD_RES 5: note = cysteine at positions 5 is in a reduced form, thus comprising as alpha-carbon side chain the structure -CH2SRx, being Rx selected independently from hydrogen (H), C1-C3-alkyl, and a protecting group
  VARIANT 6: note = X is a hydrophobic aromatic amino acids selected from phenylalanine (F), tyrosine (Y) and tryptophan (W)
  VARIANT 7: note = X is a hydrophobic amino acid selected from valine (V), leucine (L), isoleucine (I), and methionine (M)
  VARIANT 8: note = X is a basic amino acid selected from arginine (R), lysine (K), and histidine (H)
  SITE 9: note = D-Proline
  VARIANT 10: note = X is a basic amino acid selected from arginine (R), lysine (K), and histidine (H)
  VARIANT 11: note = X is a hydrophobic aromatic amino acids selected from phenylalanine (F), tyrosine (Y) and tryptophan (W)
  VARIANT 12: note = X is a hydrophobic amino acid selected from valine (V), leucine (L), isoleucine (I), and methionine (M)
  VARIANT 13: note = X is a hydrophobic amino acid selected from valine (V), leucine (L), isoleucine (I), and methionine (M)
  MOD_RES 14: note = cysteine at positions 14 is in a reduced form, thus comprising as alpha-carbon side chain the structure -CH2SRx, being Rx selected independently from hydrogen (H), C1-C3-alkyl, and a protecting group
  VARIANT 15: note = X is a hydrophobic amino acid selected from valine (V), leucine (L), isoleucine (I), and methionine (M)
  VARIANT 16: note = X is a basic amino acid selected from arginine (R), lysine (K), and histidine (H)
  VARIANT 17: note = X is a basic amino acid selected from arginine (R), lysine (K), and histidine (H)
  SITE 18: note = D-Proline
XXXXCXXXPXXXXCXXXP

SEQ ID NO: 4 - protein, 18 aa, synthetic construct
  REGION 1..18: note = Circular peptide
  DISULFID 5..14: note = intrachain disulfide bond
  SITE 9: note = D-Proline
  SITE 18: note = D-Proline
RRRLCFVRPKFVVCVRRP

SEQ ID NO: 5 - protein, 18 aa, synthetic construct
  SITE 5: note = D-Proline
  SITE 14: note = D-Proline
CVRRPRRRLCFVRPKFVV

SEQ ID NO: 6 - protein, 18 aa, Sus scrofa
  DISULFID 6..15: note = intrachain disulfide bond
  DISULFID 8..13: note = intrachain disulfide bond
RGGRLCYCRRRFCVCVGR

(19) United States
(12) Patent Application Publication
(10) Pub. No.: US 2026/0242418 A1
(43) Pub. Date: Aug. 20, 2026

(54) CYCLIC PEPTIDES WITH ANTIBACTERIAL ACTIVITY AGAINST MULTIDRUG RESISTANT (MDR) PATHOGENS

(71) Applicants: FUNDACIÓN PRIVADA INSTITUTO DE SALUD GLOBAL BARCELONA, BARCELONA (ES); UNIVERSITAT DE BARCELONA, BARCELONA (ES); FUNDACIÓ INSTITUT DE RECERCA BIOMÈDICA (IRB BARCELONA), BARCELONA (ES); HOSPITAL CLÍNIC DE BARCELONA, BARCELONA (ES)

(72) Inventors: JORDI VILA ESTAPÉ, BARCELONA (ES); CLARA BALLESTÉ DELPIERRE, BARCELONA (ES); JAVIER MORENO MORALES, BARCELONA (ES); SALVADOR GUARDIOLA BAGAN, BARCELONA (ES); ERNEST GIRALT LLEDÓ, BARCELONA (ES); NÚRIA MARTÍN VILARDELL, MOLLET DEL VALLÈS (ES)

(21) Appl. No.: 18/869,942

(22) PCT Filed: May 26, 2023
(86) PCT No.: PCT/EP2023/064218

§ 371 Date: Nov. 27, 2024

(30) Foreign Application Priority Data
May 27, 2022 (EP) 22382509.2

(51) Int. Cl. C07K 7/64; A01N 37/46; A01P 1/00; A61K 38/00; A61P 31/04

(52) U.S. Cl. CPC C07K 7/64; A01N 37/46; A01P 1/00; A61P 31/04; A61K 38/00

(57) ABSTRACT

Cyclic peptides of a particular formula (1),

(I)
(SEQ ID NO: 1)
X1X2X3X4CX6X7X8PX10X11X12X13CX15X16X17P,

wherein X1 to X17 take several meanings, and X1 and D-Pro at position 18 are linked by a peptide bond; which cyclic peptides are useful as antimicrobial agents. Bicyclic compounds, in which cysteines at positions 5 and 14 are crosslinked by a disulfide bond. Pharmaceutical compositions comprising the peptides and the medical applications of the peptides or of the compositions comprising them.

This application claims the benefit of European Patent Application EP22382509.2 filed on 27 May 2022.

### TECHNICAL FIELD

Present invention relates to the field of compounds that are active against pathogens, thus that have antimicrobial activity. It relates in particular to the field of medicine and more in particular to diseases caused by infective agents.

### BACKGROUND ART

Antimicrobial resistance (AMR) is a global health threat. International organizations such as the World Health Organization (WHO) and Centers for Disease Control and Prevention (CDC) consider it one of the most important public health challenges of our time. AMR is a natural process through which microorganisms evolve through processes such as mutations or horizontal gene transfer to become resistant to antimicrobials, such as antibacterial agents. However, the misuse and overuse of antibacterial agents in different settings (healthcare, agriculture, etc.) aided by environmental transmission are the main factors that speed up the spread of AMR.

Nosocomial infections caused by multidrug resistant bacteria (MDR) are associated with higher treatment costs, longer hospital stay and higher mortality.

The world is experiencing an increase in drug resistant pathogens, thus there is a need of novel antimicrobials, in particular antibacterial agents. To guide antimicrobial development, WHO has published a list of priority pathogens for whom development of novel and effective antimicrobials is of utmost importance. Among these, Acinetobacter baumannii, Pseudomonas aeruginosa and Enterobacterales, all Gram-negative pathogens, compose the ‘critical priority’ category. In the second category, named “high priority”, there are Gram-positive bacteria such as Enterococcus faecium and Staphylococcus aureus.

A. baumannii has been identified as an important nosocomial pathogen since the late 1970s. After developing resistance to most first line antibacterial agents, few and more toxic antibacterial agents such as colistin are the last resort for treating MDR infections caused by A. baumannii.

Protegrins are arginine-rich antimicrobial (i.e., antibacterial) peptides that belong to the cathelicidin family and are found as components of the innate immune system in many vertebrates, including humans and farm animals. These natural peptides have antimicrobial activity against bacteria, fungi, and some enveloped viruses. Despite their small size, they typically display an antiparallel β-hairpin structure that is stabilized by several disulfide bonds (See. Jang, H. et al., “Conformational Study of the Protegrin-1 (PG-1) Dimer Interaction with Lipid Bilayers and Its Effect”, BMC Struct. Biol.—2007, vol. no. 7). Among the different family members, protegrin-1 (PG-1) is the most abundant and also the most studied member of the protegrins. It consists of 18 amino acids including four cysteines that form two disulfide bonds and stabilize the β-turn peptide fold, while the N- and C-terminal tails remain more flexible. PG-1 exhibits broad-spectrum antimicrobial activity, especially against a variety of Gram-negative bacteria including MDR strains, with a mechanism consistent with membrane disruption via pore formation. However, PG-1 exhibits unfavourable drug properties, such as that it is formed by natural amino acids, and it has free N- and C-termini, which makes the molecule more susceptible to proteolytic degradation. Moreover, PG-1 also exhibits significant haemolysis and toxicity at low μM concentrations against human cells, which altogether limit its therapeutic application.

To address this issue, several synthetic PG-1 analogues have been reported, with different degrees of success, showing enhanced bactericidal activity and reduced toxicity. Among these, arguably the most notable example has been murepavadin (previously named POL7080), developed by the biotech company Polyphor. This cyclic β-hairpin peptidomimetic, with a cyclic sequence defined by cyclo[Ala-Ser-D-Pro-Pro-Thr-Trp-Ile-Dab-Orn-D-Dab-Dab-Trp-Dab-Dab](see structure below), is for example disclosed by Uper et al, in “Emerging peptide antibiotics with therapeutic potential”, Medicine in Drug Discovery 9, (2021) 100078.

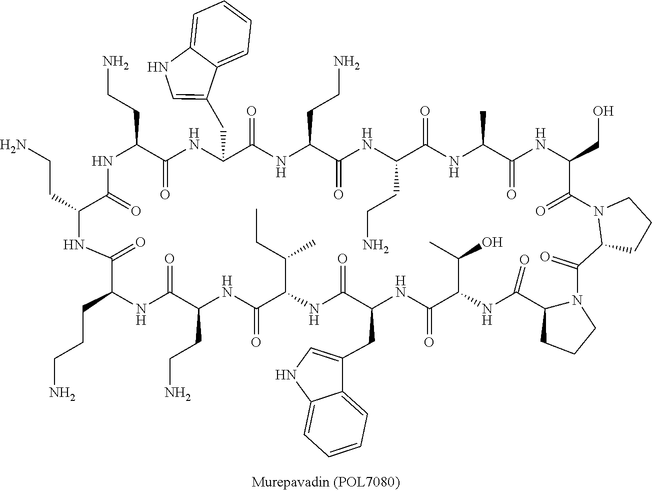 [SMILES: CC[C@H](C)[C@@H]1NC(=O)[C@H](Cc2c[nH]c3ccccc23)NC(=O)[C@H]([C@@H](C)O)NC(=O)[C@@H]2CCCN2C(=O)[C@H]2CCCN2C(=O)[C@H](CO)NC(=O)[C@H](C)NC(=O)[C@H](CCN)NC(=O)[C@H](CCN)NC(=O)[C@H](Cc2c[nH]c3ccccc23)NC(=O)[C@H](CCN)NC(=O)[C@@H](CCN)NC(=O)[C@H](CCCN)NC(=O)[C@H](CCN)NC1=O]

Murepavadin has specific and potent bactericidal activity against Pseudomonas aeruginosa, and successfully completed phase-II clinical tests in hospital patients with life-threatening Pseudomonas lung infections. However, phase-III clinical trials with intravenous administration were recently discontinued due to kidney damage in over half of the patients. Currently, a new inhaled formulation of murepavadin has been developed for treating resistant strains of P. aeruginosa in cystic fibrosis lung infections. Phase I studies has been recently completed by the biomedical company Spexis AG. Examples of additional cyclic β-hairpin peptidomimetics with a structure similar to that of murepavadin are disclosed, for example, in the patents EP1363934B31, EP1532164B31, EP1622931B1, EP2978771B1 and EP2978772B31.

Other macrocyclic peptides proposed as antimicrobials are for example disclosed in Shankaramma et al., “Macrocyclic Hairpin Mimetics of the Cationic Antimicrobial Peptide Protegrin 1: A new Family of Broad-Spectrum Antibiotics”, ChemBioChem—2002, vol. no. 3, pp.: 1126-1133. These authors also propose cyclic peptides with the template including the dimer D-Pro-Pro (i.e., one D-proline linked to an L-proline). They assay structures differing in the number of cysteines and in the number of cystine bounds (dimers or motifs). Best results are obtained with four cysteines in the cyclic structure.

Other examples of synthetic PG-1 analogues are disclosed by Tam et al. in “Membranolytic selectivity of cystine-stabilized cyclic protegrins”, Eur. J. Biochem.—2000, vol. no. 267, pp.: 3289-3300. Tam et al. disclose some cyclic compounds with a good antimicrobial activity and a relatively low haemolytic activity, in particular for compounds including at least two cysteine bonds (cystine motifs or disulfide bonds). that lead to rigid structures (see, for example the selective indexes (EC50/MIC) of ccPG3 and ccPG7).

Regarding the balance for the haemolytic activity and the antimicrobial effect of the compounds, some authors have concluded that the substitutions at certain positions in the hairpin structure, or the presence of a certain overall molecular charge and the amphipathicity are key aspects to obtain good results (i.e., selectivity for microbial cells). Nonetheless, all these substitutions are related to specific molecules, being difficult to find a pattern once a new peptide with antimicrobial activity is obtained or discovered (see for example Robinson et al. (2005), “Properties and structure-activity studies of cyclic β-hairpin peptidomimetics based on the cationic antimicrobial peptide protegrin I” Bioorganic & Medicinal Chemistry 13, 2055-2064; and Frecer et al. (2006), “QSAR analysis of antimicrobial and haemolytic effects of cyclic cationic antimicrobial peptides derived from protegrin-1”, Bioorganic & Medicinal Chemistry 14, 6065-6074).

Therefore, in spite of all efforts, there is still the need of antimicrobial agents with a high selectivity for pathogens and being non-toxic for the patient.

### SUMMARY OF INVENTION

Inventors surprisingly found new cyclic peptides with a structural pattern of sets of amino acids that differ from any of the previously disclosed PG-1 analogues. The cyclic peptides are, moreover, highly effective against a broad panel of Gram-negative and Gram-positive pathogens (i.e., bacteria), including 66 critical MDR strains, while keeping low haemolytic and cytotoxic activity in front of human cells.

The cyclic peptides of the invention comprise two cysteines and two D-prolines, being the cysteines spaced 8 amino acids each other, as well as the two D-prolines are. The cysteines are, in some particular embodiment crosslinked by a disulfide (—S—S—) bond, resulting from the oxidation of the sulfhydryl groups in the cysteine molecules. The relative position of the cysteines is important for the stabilization of the molecule.

Thus, in a first aspect the invention discloses cyclic peptides sequences of formula (I) or pharmaceutically acceptable salts thereof,

| | (I) |
|---|---|
| (SEQ ID NO: 1) | |
| | X1X2X3X4CX6X7X8PX10X11X12X13CX15X16X17P |

wherein:
Each of X1, X2, X3, X8, X10, X16 and X17 are independently a basic amino acid selected from arginine (R), lysine (K), and histidine (H);
Each of X4, X7, X12, X13 and X15 are independently a hydrophobic amino acid selected from valine (V), leucine (L), isoleucine (1), and methionine (M);
Each of X6, and X11 are independently hydrophobic aromatic amino acids selected from phenylalanine (F), tyrosine (Y) and tryptophan (WV).
Proline amino acids at position 9 and 18 are D-proline (D-Pro); and
X1 and D-Pro at position 18 are linked by a peptide bond (i.e., amide bond or amide linkage as synonymous).

These peptides defined by SEQ ID NO: 1 can also be depicted as cyclo[X1X2X3X4CX6X7X8PX10X11X12X13CX15X16X17P]. Thus, in particular the “cyclo” representing that X1 and D-Pro at position 18 are linked by a peptide bond. The cyclic peptide can also be depicted as:

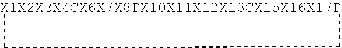

wherein the dashed line represents the peptide bond (i.e., amide linkage) between amino acid X1 and the D-Proline at position 18.

These cyclic peptides are highly effective against A. baumannii (MIC₅₀ value of 1 μg/mL and MIC₅₀ of 2 μg/mL), P. aeruginosa (MIC₅₀ value of 4 μg/mL and MIC₅₀ of 8 μg/mL) and K. pneumoniae (MIC₅₀ 4 μg/mL and MIC₅₀ 16 μg/mL). The values in parenthesis are for bicyclic peptides, thus when the cysteines at positions 5 and 14 are cross-linked to stabilize the molecule. Notwithstanding this, and as will be illustrated in examples in the next sections, cyclic peptides without the cross-linked cysteines do also fold in a β-hairpin conformation, thus they also have the antimicrobial effect. In all these molecules haemolytic activities are low, with values of IC₅₀ in haemolysis assays around 45 μg/mL (see examples in next sections).

Therefore, the cyclic peptides of the invention are proposed as active ingredients in pharmaceutical compositions, for being safe and possessing a high antibacterial activity.

It is thus a second aspect of the invention a pharmaceutical composition comprising a therapeutically effective amount of one or more of the cyclic peptides or a salt thereof, as defined in the first aspect, together with pharmaceutically acceptable excipients and/or carriers.

Directly derived from this antibacterial effect, the cyclic peptides or the pharmaceutical compositions comprising them are, in a third aspect of the invention, for use as a medicament. In other words, for use in therapy.

Also due to its antimicrobial effect the cyclic peptides are also applicable for non-therapeutic uses, such as in cosmetics or in nutrient-containing materials as preservatives, or in compositions for disinfection (disinfection of hard or soft surfaces) conveniently formulated, or in antiseptic compositions.

Thus, is another aspect of the invention the non-therapeutic use of the cyclic peptides as defined in the first aspect, as disinfectant or preservative for foodstuffs, for cosmetics, for nutrient-containing materials and for disinfectant and/or for antiseptic compositions.

Advantageously, the synthesis of the cyclic peptides of the invention is carried out by a standardized and optimized method, that takes advantage of an intramolecular native ligation reaction.

Thus, another aspect of the invention is a process for the synthesis of the cyclic peptides of the first aspect, the process comprising the following steps:
a) providing (e.g., synthesizing) a linear peptide precursor with a sequence of 18 amino acids with respective N- and C-terminal residues, which terminal residues, once ligated by a peptide bond, give a cyclic sequence defined by SEQ ID NO: 1; and
b) ligating by a peptide bond between the said respective N-, and C-terminal residues of the linear peptide of step (a) to obtain the cyclic peptide of SEQ ID NO: 1.

### BRIEF DESCRIPTION OF DRAWINGS

FIG. 1 shows a structure representation of protegrin-1 (PG-1), and of a bicyclic peptide of the invention (SEQ ID NO: 4) as a new peptidomimetic analogue (also named in this description PLP-3). PG-1 is also referred in this description as SEQ ID NO: 6 (RGGRLCYCRRRFCVCVGR, being cysteines at positions 5 and 15, and cysteines at positions 8 and 13 cross-linked by respective disulfide bonds.

FIG. 2 shows CD spectra of PLP-3 (SEQ ID NO: 4) and of Cys-reduced PLP-3 (SEQ ID NO: 5) at pH 7.4 and 25° C.

FIG. 3 shows a curve with the percentage of haemolysis of a peptide of the invention in relation to the haemolysis caused by 1% Triton X-100 (PLP-3 haemolysis % relative to 1% TX-100)

FIG. 4 shows the curve to determine the IC₅₀ for haemolysis caused by the peptide of the invention (PLP-3).

FIG. 5 (A459 human lung cancer cells) and FIG. 6 (HeLa cells), both represent the viability percentage (% viability) in relation to the logarithm of the concentration (log C (μM)) of the bicyclic peptide PLP-3. Data from a colorimetric assay with the yellow tetrazolium salt XTT (i.e., XTT cell viability assay).

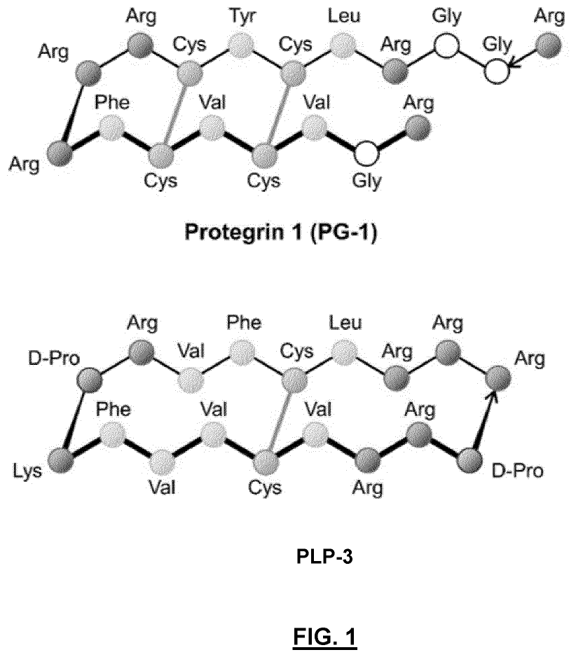

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

For the purposes of the present invention, any ranges given include both the lower and the upper end-points of the range.

If not expressly indicated, the amino acids of the cyclic peptides (either bicyclic or monocyclic intermediates) of the invention are L-amino acids.

For “amino acid” is to be understood an organic compound that contains amino and carboxylate functional groups, along with a side chain (R group) specific to each amino acid. The elements present in every amino acid are carbon (C), hydrogen (H), oxygen (O), and nitrogen (N); in addition sulfur (S) is present in the side chains of cysteine and methionine, and selenium (Se) in the less common amino acid selenocysteine. More than 500 naturally occurring amino acids are known to constitute monomer units of peptides, including proteins, (though only 20 appear in the genetic code, plus selenocysteine, which is encoded in a special way. Amino acids in the sense of the present invention include: Alanine (A), Arginine (R), Asparagine (N), Aspartic acid (Aspartate, D), Cysteine (C), Glutamine (Q), Glutamic acid (E), Glycine (G), Histidine (H), Isoleucine (I), Leucine (L). Lysine (K), methionine (M), Phenylalanine (F), Proline (P), Pyrrolysine (O), Serine (S), Selenocysteine (U), Threonine (T), Tryptophan (W), Tyrosine (Y), and Valine (V). Modified amino acids include 2-Aminoadipic acid (Aad), 3-Aminoadicpic acid (bAad), beta-Alanine (bAla), 2-Aminobutyris acid (Abu), 6-Aminocaproic acid (Acp), 2-Aminoheptanoic acid (Ahe), 2-Aminoisobutyric acid (Aib), 2-Aminopimelic acid (Apm), 2,4-Diaminobutyric acid (Dbu), Desmosine, 2, 2′-Diaminopimelic acid (Dpm), 2,3-Diaminoproprionic acid (Dpr), N-Ethylglycine (EtGly), N-Ethylasparagine (EtAsn), Hydroxylysine (Hyl), allo-Hydroxylysine (aHyl), 3-Hydroxiproline (3Hyp), 4-Hydroxyproline (4Hyp), Isodesmosine (Ide), allo-Isoleucine (alle), N-Methylisoleucine (Melle), 6-N-Methyllysine (MeLys), N-Methylvaline (Meval), Norvaline (Nva), Norleucine (Nle), and Ornithime (Orn). In several embodiments one amino acid may be replaced for another meanwhile at each selected position the hydrophobicity, aromaticity and basicity is maintained.

Amino acids used in the construction of peptides of the present invention may be prepared by organic synthesis, or obtained by other routes, such as, for example, degradation of or isolation from a natural source.

“Cyclic peptides” are polypeptide chains which contain a circular sequence of bonds. This can be through a connection between the amino and carboxyl ends (i.e., termini or residues) of the peptide, and/or; a connection between the amino end and a side chain, and/or the carboxyl end and a side chain; and/or or two side chains.

In the cyclic peptides of the invention the connection is between the amino and carboxy ends (also termed herewith N-terminal and C-terminal ends or amino and carboxy termini) of the peptide, but also in addition cyclization occurs, in particular embodiments, due to the cross-linking by disulfide bond of the two cysteines, thus it is performed with a connection of two side chains. As a result, in the later case they are, indeed, bicyclic peptides.

For the purpose of defining the cyclic peptides (i.e., amino acid sequences circular in configuration that has no amino and carboxy termini), either monocyclic or bicyclic, the amino acid in residue at position number 1 has been arbitrarily chosen, and the numbering is continuous through the entire sequence in the amino to carboxy direction.

As previously indicated, in a first aspect the invention discloses cyclic peptides sequences of formula (I) or pharmaceutically acceptable salts thereof,

| | (I) |
|---|---|
| (SEQ ID NO: 1) | |
| | X1X2X3X4CX6X7X8PX10X11X12X13CX15X16X17P |

wherein:
Each of X1, X2, X3, X8, X10, X16 and X17 are independently a basic amino acid selected from arginine (R), lysine (K), and histidine (H);
Each of X4, X7, X12, X13 and X15 are independently a hydrophobic amino acid selected from valine (V), leucine (L), isoleucine (1), and methionine (M);
Each of X6, and X11 are independently hydrophobic aromatic amino acids selected from phenylalanine (F), tyrosine (Y) and tryptophan (WV).
Proline amino acids at position 9 and 18 are D-proline (D-Pro); and
X1 and D-Pro at position 18 are linked by a peptide bond (i.e., amide bond or amide linkage as synonymous).

In a particular embodiment of the first aspect, optionally in combination with any of the embodiments below, the cyclic peptide is a bicyclic peptide of amino acid sequence (II), or a pharmaceutically acceptable salt thereof,

| | (II) |
|---|---|
| (SEQ ID NO: 2) | |
| | X1X2X3X4CX6X7X8PX10X11X12X13CX15X16X17P, |

wherein
Each of X1, X2, X3, X8, X10, X16 and X17 are independently a basic amino acid selected from arginine (R), lysine (K), and histidine (H);
Each of X4, X7, X12, X13 and X15 are independently a hydrophobic amino acid selected from valine (V), leucine (L), isoleucine (1), and methionine (M);
Each of X6, and X11 are independently hydrophobic aromatic amino acids selected from phenylalanine (F), tyrosine (Y) and tryptophan (WV).
Proline amino acids at position 9 and 18 are D-proline (D-Pro);
Cystein (C) at position 5 and 14 are linked by a disulfide bridge (i.e., disulfide bond); and
X1 and D-Pro at position 18 are linked by a peptide bond.

In a particular embodiment of the first aspect, optionally in combination with any of the embodiments below, at least three of the basic amino acids X1, X2, X3, X8, X10, X16 and X17 are arginine (R).

In another more particular embodiment at least two of the basic amino acids X1, X2, X3 and one of X16 and X17 are arginine (R). In also another more particular embodiment, at least all X1, X2, X3 and one of X16 and X17 are arginine (R).

In even a more particular embodiment of the first aspect, optionally in combination with any of the embodiments above or below, all the basic amino acids X1, X2, X3, X8, X10, X16 and X17 are arginine (R).

In another particular embodiment of the first aspect, wherein at least one of X6, and X11 is phenylalanine (F). In even a more particular embodiment, both of X6, and X11 are phenylalanine (F).

In another particular embodiment of the first aspect, optionally in combination with any of the embodiments above or below, at least one of X4, X7, X12, X13 and X15 are independently a hydrophobic amino acid selected from valine (V) and leucine (L). In a more particular embodiment, X4 is leucine (L) and X12, X13 and X15 are valine (V).

In yet a more particular embodiment, the cyclic peptide according to the first aspect in one with the amino acid sequence RRRLCFVRPKFVVCVRRP (SEQ ID NO: 4) (i.e., cyclo[RRRLCFVRPKFVVCVRRP]. This sequence is chemically represented as in Formula (III):

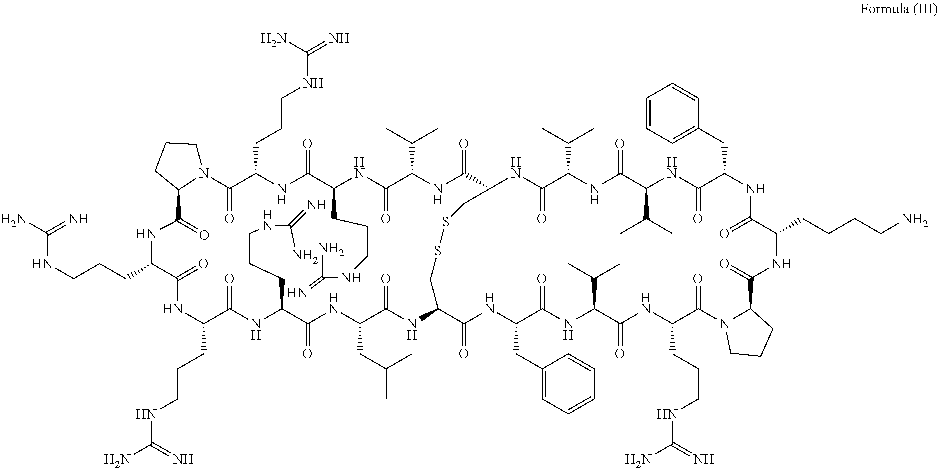 [SMILES: CC(C)C[C@@H]1NC(=O)[C@H](CCCNC(=N)N)NC(=O)[C@H](CCCNC(=N)N)NC(=O)[C@H](CCCNC(=N)N)NC(=O)[C@H]2CCCN2C(=O)[C@H](CCCNC(=N)N)NC(=O)[C@H](CCCNC(=N)N)NC(=O)[C@H](C(C)C)NC(=O)[C@@H]2CSSC[C@H](NC1=O)C(=O)N[C@@H](Cc1ccccc1)C(=O)N[C@@H](C(C)C)C(=O)N[C@@H](CCCNC(=N)N)C(=O)N1CCC[C@@H]1C(=O)N[C@@H](CCCCN)C(=O)N[C@@H](Cc1ccccc1)C(=O)N[C@@H](C(C)C)C(=O)N[C@@H](C(C)C)C(=O)N2]

This structure of Formula (III) is also disclosed in this description as the compound PLP-3. As can be seen in Formula (III), the cysteines (at positions 5 and 14 of SEQ ID NO: 4) are linked by a disulfide bond.

Considering the values of each of X1 to X17, the cyclic peptides of the invention can also be defined by referring to this SEQ ID NO: 4, and indicating a certain % of identity with said SEQ ID NO:4. Thus, the bicyclic peptides or salts thereof of the particular embodiment of the first aspect, are in a particular embodiment bicyclic peptides with a sequence that has a percentage of identity with said SEQ ID NO: 4 of at least 85%, more in particular of at least 88%, and even more in particular of at least a percentage of identity selected from 88%, 89%, 90%, 91%, 92%, 94%, 95%, 96%, 97%, 98% and 99% in relation to said SEQ ID NO: 4.

In the present invention the term “identity” refers to the percentage of residues that are identical in the two sequences when the sequences are optimally aligned. If, in the optimal alignment, a position in a first sequence is occupied by the same amino acid residue as the corresponding position in the second sequence, the sequences exhibit identity with respect to that position. The level of identity between two sequences (or “percent sequence identity”) is measured as a ratio of the number of identical positions shared by the sequences with respect to the size of the sequences (i.e., percent sequence identity=(number of identical positions/total number of positions)×100).

A number of mathematical algorithms for rapidly obtaining the optimal alignment and calculating identity between two or more sequences are known and incorporated into a number of available software programs. Examples of such programs include the MATCH-BOX, MULTAIN, GCG, FASTA, and ROBUST programs for amino acid sequence analysis, among others. Preferred software analysis programs include the ALIGN, CLUSTAL W, and BLAST programs (e.g., BLAST 2.1, BL2SEQ, and later versions thereof).

For amino acid sequence analysis, a weight matrix, such as the BLOSUM matrixes (e.g., the BLOSUM45, BLOSUM50, BLOSUM62, and BLOSUM80 matrixes), Gonnet matrixes, or PAM matrixes (e.g., the PAM30, PAM70, PAM120, PAM160, PAM250, and PAM350 matrixes), are used in determining identity.

The BLAST programs provide analysis of at least two amino acid sequences, either by aligning a selected sequence against multiple sequences in a database (e.g., GenSeq), or, with BL2SEQ, between two selected sequences. BLAST programs are preferably modified by low complexity filtering programs such as the DUST or SEG programs, which are preferably integrated into the BLAST program operations. If gap existence costs (or gap scores) are used, the gap existence cost preferably is set between about −5 and −15. Similar gap parameters can be used with other programs as appropriate. The BLAST programs and principles underlying them are further described in, e.g., Altschul et al., “Basic local alignment search tool”, 1990, J. Mol. Biol, v. 215, pages 403-410.

For multiple sequence analysis, the CLUSTAL W program can be used. The CLUSTAL W program desirably is run using “dynamic” (versus “fast”) settings. Amino acid sequences are evaluated using a variable set of BLOSUM matrixes depending on the level of identity between the sequences. The CLUSTAL W program and underlying principles of operation are further described in, e.g., Higgins et al., “CLUSTAL V: improved software for multiple sequence alignment”, 1992, CABIOS, 8(2), pages 189-191.

The cyclic peptides (monocyclic or bicyclic) of the invention include positively charged amino acid residues at the physiological pH (i.e., due to the guanidino group of the arginine side chain or the amino group in the lysine residue). Therefore, they can be in form of salts including corresponding counter-ions.

As used herein, the term “pharmaceutical acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutical acceptable salts are well known in the art. Examples of pharmaceutical acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutical acceptable salts include adipate, alginate, ascorbate, aspartate, benzenesulfonate, benzoate, bisulphate, borate, butyrate, camphorate, camphorsulfonate, citrate, cyclopentanepropionate, digluconate, dodecylsulphate, ethanesulfonate, formate, fumarate, glucoheptonate, glycerophosphate, gluconate, hemisulphate, heptanoate, hexanoate, hydroiodide, 2-hydroxy-ethanesulfonate, lactobionate, lactate, laurate, lauryl sulphate, malate, maleate, malonate, methanesulfonate, 2-naphthalenesulfonate, nicotinate, nitrate, oleate, oxalate, palmitate, pamoate, pectinate, persulphate, 3-phenylpropionate, phosphate, picrate, pivalate, propionate, stearate, succinate, sulphate, tartrate, thiocyanate, p-toluenesulfonate, undecanoate, valerate salts, and the like. Salts derived from appropriate bases include alkali metal, alkaline earth metal, and ammonium. Representative alkali or alkaline earth metal salts include sodium, lithium, potassium, calcium, magnesium, and the like. Further pharmaceutical acceptable salts include, when appropriate, nontoxic ammonium, quaternary ammonium, and amine cations formed using counterions such as halide, hydroxide, carboxylate, sulphate, phosphate, nitrate, lower alkyl sulfonate and aryl sulfonate.

The salts of the cyclic peptides or their salts of the invention may be in crystalline form either as free solvation compounds or as solvates (e.g. hydrates) and it is intended that both forms are within the scope of the present invention. Methods of solvation are generally known within the art. As above exposed particular solvates include hydrated salts, more in particular mono- and dihydrated sals, as well as esters of said salts.

These peptides are, in some embodiments, conjugated to a cargo. Thus, herewith disclosed are conjugates comprising:
a cyclic peptide as defined in the first aspect and/or in any of its embodiments; and
a cargo.

Examples of cargos include other peptides, nucleic acids, proteins, polysaccharides, lipids, lipoproteins, glycolipids, small molecules and mixtures thereof. In addition, the cargo comprises in other examples vesicles (such as nanovesicles) from the group of micelles, liposomes, extracellular vesicles, nanoemulsions, quantum dots, dendrimers and mixtures thereof.

These cargos allow the delivery to desired targets (i.e., cells). There, the peptides of the invention can perform their effect as antimicrobial agents.

The peptides are, as will be illustrated in the examples, active against a broad spectrum of microorganisms, thus they are conceived as active ingredients in several compositions including other ingredients.

Thus, the second aspect of the invention refers to pharmaceutical compositions comprising a therapeutically effective amount of the cyclic peptides, and/or a salt thereof, as defined in the first aspect, together with pharmaceutically acceptable excipients and/or carriers.

The term “pharmaceutical” encompasses also the concept of “veterinary composition”. Thus, they relate to compositions that are therapeutically effective when administered by any desired or applicable route to any animal, including humans.

The expression “therapeutically effective amount” as used herein, refers to the amount of a compound that, when administered, is sufficient to prevent development of, or alleviate to some extent, one or more of the symptoms of the disease which is addressed. The particular dose of compound administered according to this invention will of course be determined by the particular circumstances surrounding the case, including the compound administered, the route of administration, the particular condition being treated, and the similar considerations.

The expression “pharmaceutically acceptable excipients or carriers” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio. Examples of suitable acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In a particular embodiment of the pharmaceutical compositions of the invention, they comprise one or more of the cyclic peptides of the invention, or one or more of their salts, or a combination of the one or more cyclic peptides and one or more salts thereof.

In a particular embodiment of the compositions of the second aspect, they are in a form for administration selected from the group consisting of oral, topical, injectable (including transdermal, subcutaneous and intravenous injections), buccal, rectal, transmucosal, and combinations thereof.

In another particular embodiment of the compositions of the second aspect, they are solid compositions, in particular selected from tablets, pills, capsules, granules, chewing gums; liquid compositions, in particular selected from suspensions and solutions, or compositions in the form of a gel, cream, ointment, nebulizers, ovules or suppositories.

Additionally, the compositions of the present invention may contain other ingredients, such as fragrances, colorants, and other components known in the state of the art for use in any of the selected routes of administration to provide the proper galenic formulations.

The formulations of the pharmaceutical compositions described herein may be prepared by any method known or hereafter developed in the art of pharmacology. In general, such preparatory methods include the step of bringing the active ingredient (the peptide) into association with a excipient and/or one or more other accessory ingredients, and then, if necessary and/or desirable, shaping and/or packaging the product into a desired single- or multi-dose unit.

A pharmaceutical composition of the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient.

The relative amounts of the active ingredient (i.e., the mono- or bicyclic peptide or salt thereof as defined in any of the previous aspects and embodiments), the acceptable excipients, and/or any additional ingredients in the composition of the invention will vary, depending upon the identity, size, and/or condition of the subject treated and further depending upon the route by which the composition is to be administered. Namely, they are manufactured by means of conventional mixing, dissolving, granulating, coated tablet-making, levigating, emulsifying, encapsulating, entrapping or lyophilizing processes.

Acceptable excipients used in the manufacture of these compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulphate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulphate, quaternary ammonium compounds, and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminium silicate] and Veegum [magnesium aluminium silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters {e.g., polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g., polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g., Cremophor), polyoxyethylene ethers, (e.g., polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulphate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinylpyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulphite, propionic acid, propyl gallate, sodium ascorbate, sodium bisulphite, sodium metabisulphite, and sodium sulphite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerine, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulphate (SLS), sodium lauryl ether sulphate (SLES), sodium bisulphite, sodium metabisulphite, potassium sulphite, potassium metabisulphite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminium hydroxide, alginic acid, pyrogen-free water, isotonic saline, Ringer's solution, ethyl alcohol, and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulphate, sodium lauryl sulphate, and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for parenteral administration include, but are not limited to, pharmaceutically acceptable liposomes emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, the conjugates of the invention are mixed with solubilizing agents such as polyethoxylated castor oil (e.g., CREMOPHOR™), alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. Alternatively, the preparation can be in the form of liposomes.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

The peptides of the invention can be in micro-encapsulated form with one or more excipients as noted above. In one embodiment, the peptides of the invention are formulated in liposomes.

The pharmaceutical composition of the present invention may be administered either alone or in combination with one or more other therapeutic agents. By “in combination with,” it is not intended to imply that the agents must be administered at the same time and/or formulated for delivery together, although these methods of delivery are within the scope of the invention. The compositions can be administered concurrently with, prior to, or subsequent to, one or more other desired therapeutics or medical procedures. In general, each agent will be administered at a dose and/or on a time schedule determined for that agent. Additionally, the invention encompasses the delivery of the peptide or pharmaceutical compositions in combination with agents that may improve their bioavailability, reduce and/or modify their metabolism, inhibit their excretion, and/or modify their distribution within the body.

Thus, there are also herewith disclosed therapeutic combinations of compounds comprising: (a) a therapeutically effective amount of the peptide of formula (I) (i.e., the monocyclic peptide of SEQ ID NO: 1 or the bicyclic peptide of SEQ ID NO: 2), or a salt thereof, and (b) one or more other therapeutic agents, in particular antimicrobial agents. Examples of antimicrobial agents include, in particular, one or more of the biostatic agents, Chloramphenicol, Clindamycin, Ethambutol, Lincosamides, Macrolides, Nitrofurantoin, Novobiocin, Oxazolidinone, Spectinomycin, Sulfonamides, Tetracyclines, Tigecycline and Trimethoprim. In another particular embodiment, the one or more other therapeutic agents are selected from the antibacterial agents Vancomycin, Teicoplanin, Linezolid, Daptomycin, Trimethoprim/sulfamethoxazole, Doxycycline, Ceftobiprole, Ceftaroline, Dalbavancin, Fusidic acid, Mupirocin, Omadacycline, Oritavancin, Tedizolid, Telavancin, Tigecycline, Aminoglycosides, Carbapenems, Ceftazidime, Cefepime, Ceftolozane/tazobactam, Fluoroquinolones, Piperacillin/tazobactam, Ticarcillin/clavulanic acid, and Streptogramins.

The particular combination of therapies to employ in a combination regimen will take into account compatibility of the desired therapeutics and/or procedures and/or the desired therapeutic effect to be achieved. It will be appreciated that the therapies employed may achieve a desired effect for the same disorder (for example, a peptide of the invention may be administered concurrently with another biologically active agent used to treat the same disorder), and/or they may achieve different effects (e.g., control of any adverse effects). In will further be appreciated that biologically active agents utilized in this combination may be administered together in a single composition or administered separately in different compositions.

The expression “in combination with” also encompasses the possibility of conjugating (by chemical-physical interactions) the peptide of the invention to any of the further agents mentioned above and below, which can be either a therapeutic agent or an agent for improving the profile of the peptide (such as bioavailability), among others.

The peptide or a salt thereof, or the pharmaceutical composition comprising it as defined in any one of the previous aspects are, thus, for use as a medicament (i.e., in therapy) as a third aspect of the invention.

In a more particular embodiment, either the cyclic peptide and/or a salt thereof, or the pharmaceutical composition comprising one or more of them are for use in the prevention and/or treatment of a disease caused by a pathogen. Thus, either the cyclic peptide and/or a salt thereof, or the pharmaceutical composition comprising one or more of them are for use as a pharmaceutically active substance having antimicrobial (e.g., antibiotic) activity.

This embodiment can also be formulated as the use of either the cyclic peptide and/or a salt thereof, or the pharmaceutical composition comprising a therapeutically effective amount of one or more of them, for the preparation of a medicament for the prevention and/or treatment of a disease caused by a pathogen. The invention relates also to a method for the prophylaxis (prevention) and/or treatment of a disease caused by a pathogen, comprising administering a therapeutically effective amount of a cyclic peptide and/or a salt thereof, or a pharmaceutical composition comprising one or more of them, all as defined above, together with pharmaceutically acceptable excipients or carriers, in a subject in need thereof, including a human.

In a more particular embodiment of the cyclic peptide or a salt thereof, or the pharmaceutical composition, for the use as defined above, the pathogen causing disease is a Gram-negative bacteria, more in particular selected from the group consisting of Pseudomonas aeruginosa, Acinetobacter baumannii, and Klebsiella pneumoniae.

In a more particular embodiment of the cyclic peptide or a salt thereof, or the pharmaceutical composition, for the use as defined above, the pathogen causing disease is a Gram-positive bacteria, more in particular selected from the group consisting of Staphylococcus aureus, Enterococcus faecium, and Enterococcus faecalis.

In another particular embodiment, the cyclic peptide (i.e., mono- or bicyclic) or a salt thereof, or the pharmaceutical composition comprising it, for use as defined above in the previous third aspect and embodiments, is administered in combination with another therapeutic agent, as previously indicated.

This “another active agent or therapeutic agent” is in particular selected from one or more compounds with antimicrobial activity. In particular they are antibacterial compounds, such as biocidal compounds or biostatic compounds.

Also as previously indicated, the said administration in combination can be performed with the disclosed pharmaceutical combinations.

In also another particular embodiment of the third aspect of the invention, optionally in combination with any of the embodiments above or below, the cyclic peptide or a salt thereof, or the pharmaceutical composition, is for use in in the prevention and/or treatment of an infectious disease, or a disease or condition associated to this infectious disease.

Particular embodiments of an infectious disease, or a disease or condition associated to this infectious disease are selected from the group consisting of infections related to respiratory diseases, or skin or soft tissue diseases, or gastrointestinal diseases, or eye diseases, or ear diseases or CNS diseases, or bone diseases, or cardiovascular diseases or genitourinary diseases, or nosocomial infections, or catheter-related and non-catheter-related infections, or urinary tract infections, or bloodstream infections.

More particular embodiments of the infections related to respiratory diseases are selected from the group consisting of infections associated to cystic fibrosis, emphysema and asthma. More particular embodiments of the infections related to skin or soft tissue diseases are selected from infections associated to surgical wounds, traumatic wounds or burn wounds. More particular embodiments of the infections related to gastrointestinal diseases are selected from the group consisting of infections causing diarrhoea, infections associated to necrotizing enterocolitis or typhlitis. More particular embodiments of the infections related to eye diseases are selected from infectious keratitis or endophthalmitis. More particular embodiments of the infections related to ear diseases include infections causing otitis. More particular embodiments of the infections related to CNS diseases are selected from the group consisting of infections associated to brain abscess or meningitis. More particular embodiments of the infections related to bone diseases are selected from the group consisting of infections accompanying osteochondritis or osteomyelitis. More particular embodiments of the infections related to cardiovascular diseases are selected from the group consisting of endocarditis or pericarditis. More particular embodiments of the infections related to gastrourinal diseases are selected from epididymitis, prostatitis or urethritis.

In another more particular embodiment, optionally in combination with the embodiments of the third aspect of the invention, the cyclic peptide or a salt thereof, or the pharmaceutical composition, are for use in the treatment of an infection, especially nosocomial infections, such as urinary tract infections, bloodstream infections, ventilator-associated pneumonia (VAP), hospital-acquired pneumonia (HAP), and healthcare-associated pneumonia (HCAP).

Besides their therapeutic activity in case of disease of a plant or animal, the cyclic peptides, and their salts are effective and applicable in many fields due to its capacity of inhibiting the growth or of killing microorganisms, in particular of Gram-negative and Gram-positive bacteria. Thus, as indicated, another aspect of the invention relates to non-therapeutic uses of the cyclic peptide as defined the first aspect, as disinfectant or preservative for foodstuffs, for cosmetics, and for nutrient-containing materials. For use as disinfectants or preservatives one or more of the cyclic peptides and/or their salts can be added to the bulk of compositions even comprising additional and different antimicrobial agents. Also as previously indicated, the cyclic peptides, and their salts are used as antiseptic agents in antiseptic compositions.

In the particular case of avoiding reproduction of the microorganisms, the cyclic or bicyclic peptides act as inhibitors of biofilm formation. Biofilms are glycocalyx-containing materials secreted by individual microorganisms in which are encased communities of these microorganisms. Biofilms allow these microorganisms to adhere to a solid surface and be enveloped within a protective extracellular glycocalyx-containing matrix.

Due to its ability to avoid or prevent biofilm formation, the cyclic or bicyclic peptides of the invention are, in another particular embodiment useful as anti-biofilm forming agents in surfaces. Examples of surfaces where it is noteworthy the avoidance of microorganism biofilm formation are surgical or medical instrumentation. The surgical or medical instrumentation is, in particular selected from stents (e.g., cardiovascular stents, balloons), scalpels, probes (e.g., urinary probes), and implants.

Thus, it is also herewith disclosed a medical device that comprises a coating, said coating comprising a cyclic or bicyclic peptide and/or a salt thereof as defined in the previous aspect, or a composition comprising the same. Examples of such medical devices are, as indicated, stents (e.g., cardiovascular stents, balloons), scalpels, probes (e.g., urinary probes), and implants.

Also due to the biofilm formation inhibition, the cyclic or bicyclic peptides of the invention are for use in diseases caused by microorganism biofilm formation. In a particular embodiment, the diseases caused by microorganism biofilm formation are selected from cystic fibrosis, acne, chronic bacterial prostatitis, urinary tract infections, otitis media, infective endocarditis, atherosclerosis, sialolithiasis, recalcitrant typhoid fever and predisposition to hepatobiliary cancers, and inflammatory bowel disease, wound infections, bacterial vaginosis, mastitis, chronic rhinosinusitis, pharyngitis and laryngitis, and combinations thereof.

The peptides as defined in the first aspect are prepared by a process for their synthesis comprising the steps of:
a) providing (e.g., synthesizing) a linear peptide precursor with a sequence of 18 amino acids with respective N- and C-terminal residues, which terminal residues once ligated by a peptide bond give a cyclic sequence defined by SEQ ID NO: 1; and
b) ligating by a peptide bond the said respective N-, and C-terminal residues of the linear peptide of step (a) to obtain the cyclic peptide of SEQ ID NO: 1.

In the particular case of the bicyclic peptides of SEQ ID NO: 2, disclosed in particular embodiments of the first aspect, the invention encompasses also a process for their synthesis comprising the following steps:
(a) providing a cyclic (i.e., monocyclic) peptide sequence defined by SEQ ID NO: 3:

| (SEQ ID NO: 3) | |
|---|---|
| | X1X2X3X4CX6X7X8PX10X11X12X13CX15X16X17P, |

wherein each of X1 to X17 have the same meaning as indicated for the peptides of the first aspect and its embodiments, prolines at positions 9 and 18 are D-Pro, cysteines at positions 5 and 14 are in a reduced form, thus comprising as α-carbon side chain the structure —CH2SR^{x}, being R^{x} selected independently from hydrogen (H), C1-C3-alkyl, or a protecting group, and being X1 and D-Pro at position 18 linked by a peptide bond; and
(b) providing the bicyclic peptide of SEQ ID NO: 2 as the particular embodiment of the first aspect, from the cyclic peptide of SEQ ID NO: 3.

In other words, the process for these bicyclic peptides is defined in more detail by the following steps
(a) providing (e.g., synthesizing) a linear peptide precursor with a sequence of 18 amino acids with respective N- and C-terminal residues, which terminal residues once ligated by a peptide bond give a cyclic sequence defined by SEQ ID NO: 3:

| (SEQ ID NO: 3) | |
|---|---|
| | X1X2X3X4CX6X7X8PX10X11X12X13CX15X16X17P, |

wherein each of X1 to X17 have the same meaning as indicated above for the first aspect, prolines at positions 9 and 18 are D-Pro, and being cysteines at positions 5 and 14 in a reduced form, thus comprising as α-carbon side chain the structure —CH₂SR^{x}, being R^{x} selected independently from H, C1-C3-alkyl, or a protecting group;
(b) ligating by a peptide bond the said respective N-, and C-terminal residues of the linear peptide of step (a) to obtain the cyclic peptide of SEQ ID NO: 3; and
(c) linking (e.g., by oxidizing) the cysteines at position 5 and 14 of SEQ ID NO: 3 to obtain the bicyclic peptide of SEQ ID NO: 2.

Step of the provision of a linear peptide precursor can be started from any of the 18 amino acids of SEQ ID NO: 1 (or 2 or 3) to obtain the linear peptide. By this way, several linear peptides are synthesized, all of them including N- and C-terminal residues which, once ligated, always result in the particular order from N- to C-terminal as indicated in any of the cyclic sequences SEQ ID NO: 1, or 2, or 3.

This means that, in a particular embodiment of the process, step (a) is carried out by: (a1) providing a linear peptide precursor with a sequence of 18 amino acids as defined in SEQ ID NO: 3, wherein the amino acid at the N-terminal residue is one selected from any of the 18 amino acids in said SEQ ID NO: 3, and the remaining amino acids are those disposed, or ordered, in the amino to carboxy direction as defined in SEQ ID NO: 3, being the C-terminal residue the amino acid at position 18 from the selected N-terminal (counting as amino acid at position 1) in the said carboxy direction; and (a2) cycling the linear peptide ligating by a peptide bond said respective N-, and C-terminal residues of the linear peptide of step (a1) to obtain the cyclic peptide of SEQ ID NO: 3.

This means that if the amino acid X4 is the N-terminal residue in the linear peptide precursor, the C-terminal residue in this linear peptide is the amino acid X3 of any of the cyclic sequences SEQ ID NO: 1, or 2, or 3. Or if the N-terminal residue in the linear peptide precursor is the cysteine (C) at position 14, the C-terminal residue in the linear precursor is the amino acid at position X13 of any of the cyclic sequences SEQ ID NO: 1, or 2, or 3.

In other words, the process for the synthesis of the bicyclic peptides of the embodiment of the first aspect can also be defined by:
(a) providing a linear peptide precursor with a sequence of 18 amino acids with respective N- and C-terminal residues, which residues once ligated by a peptide bond give a cyclic sequence defined by SEQ ID NO: 3:
X1X2X3X4CX6X7X8PX10X11X12X13CX15X16X17P (SEQ ID NO: 3), wherein each of X1 to X17 have the same meaning as indicated above for the first aspect, prolines at positions 9 and 18 are D-Pro have the same meaning as indicated above for the first aspect, X1 and D-Pro at position 18 are linked by a peptide bond, and being cysteines at positions 5 and 14 in a reduced form, thus comprising as α-carbon side chain chain the structure —CH2SR^{x}, being R^{x} selected independently from hydrogen (H), C1-C3-alkyl, or a protecting group, and being X1 and D-Pro at position 18 linked by a peptide bond;
(b) ligating by a peptide bond the said respective N-, and C-terminal residues of the linear peptide of step (a) to obtain the SEQ ID NO: 3; and
(c) linking the cysteines at position 5 and 14 of SEQ ID NO: 3 to obtain the bicyclic peptide of SEQ ID NO: 2.

When it is indicated that the SEQ ID NO: 3 or any other is provided, it means that the precursor linear peptide is synthesized by any of the means known by the skilled person of the art, including for example the solid-phase protein synthesis. The peptides, and in particular the linear precursors or fragments thereof are in other embodiments provided by gene recombinant technology in organisms capable of being effective synthesizers of peptides including D-amino acids (as for the D-Pro in the sequences), or for example of providing fragments of the said linear peptide precursors, and then finishing their synthesis and ligation by any synthetic method allowing the introducing of D-Prolines at the specific positions of the fragments of the linear peptide. In any case, the peptides are synthetic constructs (i.e., non-naturally occurring).

In a particular embodiment of the process, optionally in combination with any of the embodiments of the process previously disclosed, the step of linking the cysteines at position 5 and 14 of SEQ ID NO: 3 to obtain the cyclic peptide of SEQ ID NO: 2 is carried out by any of the methods known by the skilled person in the art, such us by oxidizing the cysteines at position 5 and 14 of SEQ ID NO: 3 to obtain the cyclic peptide of SEQ ID NO: 2. This oxidizing (or linking step) is preceded first or simultaneously by deprotecting the cysteine residues, if protected. The linking, for example by oxidation, leads to the formation of the disulfide bond between the cysteines.

In a particular embodiment of the process of synthesis, step (a) comprises an Fmoc-based solid-phase peptide synthesis (SPPS) of the linear precursor, thus in particular said precursor comprising in its C-terminal end an N-acylurea moiety. This moiety acts as thioester precursor during the ligation of N- and C-terminal residues of the linear precursor peptide to obtain the cyclic peptide.

The general principle of solid phase peptide synthesis is to repeat cycles of deprotection-wash-coupling-wash. The free N-terminal amine of a solid-phase attached peptide is coupled to a single N-protected amino acid unit. This unit is then deprotected, revealing a new N-terminal amine to which a further amino acid may be attached. Amino acids have reactive moieties at the N- and C-termini, which facilitates amino acid coupling during synthesis. Many amino acids also have reactive side chain functional groups, which can interact with free termini or other side chain groups during synthesis and peptide elongation and negatively influence yield and purity. To facilitate proper amino acid synthesis with minimal side chain reactivity, chemical groups have been developed to bind to specific amino acid functional groups and block, or protect, the functional group from nonspecific reactions. These protecting groups, while vast in nature, can be separated into three groups, as follows: N-terminal protecting groups, C-terminal protecting groups (mostly used in liquid-phase synthesis), and side chain protecting groups.

For coupling the peptides, the carboxyl group is usually activated. This is important for speeding up the reaction. There are two main types of activating groups: carbodiimides and triazolols. However, the use of pentafluorophenyl esters (FDPP, PFPOH]) and BOP—Cl are useful for cyclising peptides.

Purified, individual amino acids are reacted with these protecting groups prior to synthesis and then selectively removed during specific steps of peptide synthesis.

Exemplary resins which may be employed by the present invention include, but are not limited to: (1) alkenyl resins (e.g., REM resin, vinyl sulfone polymer-bound resin, vinyl-polystyrene resin); (2) amine functionalized resins (e.g., amidine resin, N-(4-Benzyloxybenzyl)hydroxylamine polymer bound, 3-(Fmoc-amino)-4-aminobenzoyl AM resin, (aminomethyl)polystyrene, polymer bound (R)-(+)-a-methylbenzylamine, 2-Chlorotrityl Knorr resin, 2-N-Fmoc-Amino-dibenzocyclohepta-1,4-diene, polymer-bound resin, 4-[4-(1-Fmoc-aminoethyl)-2-methoxy-5-nitrophenoxy]butyramidomethyl-polystyrene resin, 4-Benzyloxybenzylamine, polymer-bound, 4-Carboxybenzenesulfonamide, polymer-bound, Bis(tert-butoxycarbonyl)thiopseudourea, polymer-bound, Dimethylaminomethyl-polystyrene, Fmoc-3-amino-3-(2-nitrophenyl)propionic acid, polymer-bound, N-Methyl aminomethylated polystyrene, PAL resin, Sieber amide resin, tert-Butyl N-(2-mercaptoethyl)carbamate, polymer-bound, Triphenylchloromethane-4-carboxamide polymer bound); (3) benzhydrylamine (BHA) resins (e.g., 2-Chlorobenzhydryl chloride, polymer-bound, HMPB-benzhydrylamine polymer bound, 4-Methylbenzhydrol, polymer-bound, Benzhydryl chloride, polymer-bound, Benzhydrylamine polymer-bound); (4) Br-functionalized resins (e.g., 4-(Benzyloxy)benzyl bromide polymer bound, 4-Bromopolystyrene, Brominated PPOA resin, Brominated Wang resin, Bromoacetal, polymer-bound, Bromopolystyrene, HypoGel® 200 Br, Polystyrene A-Br for peptide synthesis, Selenium bromide, polymer-bound, TentaGel HL-Br, TentaGel MB-Br, TentaGel S-Br); (5) Chloromethyl resins (e.g., 5-[4-(Chloromethyl)phenyl]pentyl]styrene, polymer-bound, 4-(Benzyloxy)benzyl chloride polymer bound, 4-Methoxybenzhydryl chloride, polymer-bound); (6) CHO-functionalized resins (e.g., (4-Formyl-3-methoxyphenoxymethyl)polystyrene, (4-Formyl-3-methoxyphenoxymethyl)polystyrene, 3-Benzyloxybenzaldehyde, polymer-bound, 4-Benzyloxy-2,6-dimethoxybenzaldehyde, polymer-bound, Formylpolystyrene, HypoGel® 200 CHO, Indole resin, Polystyrene A-CH(OEt)2, TentaGel HL-CH(OEt)2); (7) Cl-functionalized resins (e.g., Benzoyl chloride polymer bound, (chloromethyl)polystyrene, Merrifield's resin); (8) CO2H functionalized resins (e.g., Carboxyethylpolystryrene, HypoGel® 200 COOH, Polystyrene AM-COOH, TentaGel HL-COOH, TentaGel MB—COOH, TentaGel S—COOH); (9) Hypo-Gel resins (e.g., HypoGel® 200 FMP, HypoGel® 200 PHB, HypoGel® 200 Trt-OH, HypoGel® 200 HMB); (10) I-functionalized resins (e.g., 4-Iodophenol, polymer-bound, Iodopolystyrene); Janda-Jels™ (JandaJel<ä>-Rink amide, JandaJel-NH2, JandaJel-Cl, JandaJel-4-Mercaptophenol, JandaJel-OH, JandaJel-1-(3-Dimethylaminopropyl)-3-ethylcarbodiimide, JandaJel-1,3,4,6,7,8-hexahydro-2H-pyrimido-[1,2-a]pyrimidine, JandaJel-morpholine, JandaJel-polypyridine, JandaJel-Triphenylphosphine, JandaJel-Wang); (11) MBHA resins (3[4′-(Hydroxymethyl)phenoxy]propionic acid-4-methylbenzhydrylamine resin, 4-(Hydroxymethyl)phenoxyacetic acid polymer-bound to MBHA resin, HMBA-4-methylbenzhydrylamine polymer bound, 4-Methylbenzhydrylamine hydrochloride polymer bound Capacity (amine)); (12) NH2 functionalized resins ((Aminomethyl)polystyrene, (Aminomethyl)polystyrene, HypoGel® 200 NH2, Polystyrene AM-NH2, Polystyrene Microspheres 2-aminoethylated, Polystyrol Microspheres 2-bromoethylated, Polystyrol Microspheres 2-hydroxyethylated, TentaGel HL-NH2, Tentagel M Br, Tentagel M NH2, Tentagel M OH, TentaGel MB-NH2, TentaGel S—NH2, TentaGel S—NH2); (13) OH-functionalized resins (e.g., 4-hydroxymethylbenzoic acid, polymer-bound, Hydroxymethyl Resins, OH-functionalized Wang Resins); (14) oxime resins (e.g., 4-Chlorobenzophenone oxime polymer bound, Benzophenone oxime polymer bound, 4-Methoxybenzophenone oxime polymer bound); (15) PEG resins (e.g., ethylene glycol polymer bound); (16) Boc-/Blz peptide synthesis resins (e.g., Boc-Lys(Boc)-Lys[Boc-Lys(Boc)]-Cys(Acm)-b-Ala-O-PAM resin, Boc-Lys(Fmoc)-Lys[Boc-Lys(Fmoc)]-b-Ala-O-Pam resin, Boc-Lys(Boc)-Lys[Boc-Lys(Boc)]-Lys{Boc-Lys(Boc)-Lys[Boc-Lys(Boc)]}-b-Ala-O-PAM resin, Boc-Lys(Fmoc)-Lys[Boc-Lys(Fmoc)]-Lys[Boc-Lys(Fmoc)-Lys{Boc-Lys(Fmoc)]}-b-Ala-O-PAM resin, Boc-Lys(Boc)-Lys[Boc-Lys(Boc)]-Lys{Boc-Lys(Boc)-Lys[Boc-Lys(Boc)]}-Cys(Acm)-b-Ala-O-PAM resin, Preloaded PAM resins); (17) Fmoc-/t-Bu peptide synthesis resins (e.g., Fmoc-Lys(Fmoc)-Lys[Fmoc-Lys(Fmoc)]-b-Ala-O-Wang resin, Fmoc-Lys(Fmoc)-Lys[Fmoc-Lys(Fmoc)]-Lys{Fmoc-Lys(Fmoc)-Lys[Fmoc-Lys(Fmoc)]}-b-Ala-O-Wang resin, Preloaded TentaGel® S Trityl Resins, Preloaded TentaGel® Resins, Preloaded Trityl Resins, Preloaded Wang Resins, Trityl Resins Preloaded with Amino Alcohols); (19) thiol-functionalized resins (e.g., HypoGel® 200 S-Trt, Polystyrene AM-S-Trityl, TentaGel HL-S-Trityl, TentaGel MB-S-Trityl, TentaGel S—S-Trityl); and (20) Wang resins (e.g., Fmoc-Ala-Wang resin, Fmoc-Arg(Pbf)-Wang resin, Fmoc-Arg(Pmc)-Wang resin, Fmoc-Asn(Trt)-Wang resin, Fmoc-Asp(OtBu)-Wang resin, Fmoc-Cys(Acm)-Wang resin, Fmoc-Cys(StBu)-Wang resin, Fmoc-Cys(Trt) Wang resin, Fmoc-Gln(Trt)-Wang resin, Fmoc-Glu(OtBu)-Wang resin, Fmoc-Gly-Wang resin, Fmoc-His(Trt)-Wang resin, Fmoc-Ile-Wang resin, Fmoc-Leu-Wang resin, Fmoc-Lys(Boc)-Wang resin, Fmoc-Met-Wang resin, Fmoc-D-Met-Wang resin, Fmoc-Phe-Wang resin, Fmoc-Pro-Wang resin, Fmoc-Ser(tBu)-Wang resin, Fmoc-Ser(Trt)-Wang resin, Fmoc-Thr(tBu)-Wang resin, Fmoc-Trp(Boc) Wang resin, Fmoc-Trp-Wang resin, Fmoc-Tyr(tBu)-Wang resin, Fmoc-Val-Wang resin).

“Protecting group” (PG) refers to a grouping of atoms that when attached to a reactive group in a molecule masks, reduces or prevents that reactivity.

Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (lpaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, Ntriphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), p-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitable thiol-protecting groups, in particular when referring to Rx, are selected from the group consisting of acetamidomethyl (Acm), tert-butyl (But), 3-nitro-2-pyridine sulfenyl (NPYS), 2-pyridine-sulfenyl (Pyr), and trityl (Trt) groups.

In another particular embodiment of the process for the synthesis of the cyclic or bicyclic peptides of the invention, step of ligating by a peptide bond, the said respective N-, and C-terminal residues of the linear peptide precursor to obtain the SEQ ID NO: 3 or SEQ ID NO: 1 is carried out by:
(i) dissolving the linear precursor peptide with a ligation buffer at a pH from 6.5 to 8.5 in particular at pH 7, the said buffer comprising a guanidinium salt as chaotropic agent (i.e., as compound aiding the denaturation of the peptide structure), such as guanidinium hydrochloride, and let the solution under agitation at room temperature (i.e., 18-25° C.), and inert atmosphere, in particular with a gas selected from nitrogen and argon, for a period to allow the ligation, in particular from 1 to 5 hours, more in particular 4 hours; (ii) acidifying and extracting the mixture of (i) with an organic non-polar solvent comprising, in particular, tert-butyl methyl ether;
(iii) desalting the acidified and extracted solution, in particular with a cartridge for desalting, although other means are also applicable, to obtain separated the guanidinium salts and the cyclic (i.e., ligated) peptide; and
(iv) optionally, in the particular case when a cartridge for desalting is used eluting the cyclic peptide with a solvent comprising water and an organic solvent selected in particular from acetonitrile and methanol, in particular at a 1:1 ratio of both co-solvents water:organic solvent.

A particular ligation buffer comprises the guanidinium salt at a concentration in the buffer from 2 to 8 M, in particular 6M; a phosphate salt, in particular an alkaline or alkaline earth phosphate salt, such as sodium phosphate, being the phosphate salt at a concentration in the ligation buffer from 150 mM to 250 mM, in particular at 200 mM; and one or more reducing agents, in particular selected from Tris(2-carboxyethyl)phosphine (TCEP), and a thiol group-containing compound which acts also as a catalyst, in particular 4-mercaptophenol and mixtures thereof. When (TCEP) is present, the concentration in the ligation buffer is from 2 to 500 mM, in particular 20 mM. When 4-mercaptophenol is present, the concentration in the ligation buffer is from 2 to 500 mM, in particular it is 100 mM.

In another particular embodiment, the linear precursor peptide is dissolved in the ligation buffer at a concentration comprised from 0.1 to 100 mM, in particular from 2 to 3 mM.

In another particular embodiment of the process for the synthesis of the bicyclic peptide of the invention, step of linking the cysteines at position 5 and 14 of SEQ ID NO: 3 to obtain the bicyclic peptide of SEQ ID NO: 2, is carried out by cysteine oxidation by first preparing an aqueous solution of the cyclic peptide of SEQ ID NO: 3 at a concentration from 0.5 to 500 μM, in particular from 20-40 μM (i.e., highly diluted conditions) and at a pH from 7.0 to 8.5 with any appropriate buffer, and stirring under air oxygen for a period to allow the formation of the intramolecular disulfide bond, in particular for a period of 24 hours; and further separating the cyclic peptide with the disulfide bond from the oxidation solution.

The separation of the bicyclic peptide with the disulfide bond obtained after the cysteine linking step is carried out by any separation technique known by the skilled person in the art.

In another particular embodiment of the process, the separation of bicyclic peptide with the disulfide bond is carried out by chromatography, in particular by high performance liquid chromatography (HPLC), more in particular by semi-preparative HPLC, and using as mobile phase a first solvent comprising water and TFA, in particular the trifluoroacetic acid (TFA) at 0.1%, and a second solvent comprising TFA, in particular at 0.1% in acetonitrile (ACN). By this way the cyclic peptide, which indeed is a bicyclic peptide due to the disulfide bond is obtained with the desired purity degree (i.e., 95%).

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

### Example 1. Solid-Phase Cyclic Peptide Synthesis and Intramolecular Chemical Ligation

The cyclic peptide of SEQ ID NO: 4 (also named PLP-3 bicyclic peptide) was synthesized on Dawson Dbz AM resin (Merck Millipore) with a substitution of 0.4-0.5 mmol/g.

First a linear peptide precursor of SEQ ID NO: 5 (CVRRPRRRLCFVRPKFVV) was synthesized as follows (due to the synthetic route in this linear sequence D-Prolines are at positions 5 and 14, respectively):

A first amino acid (Val in the C-terminal of SEQ ID NO: 5) was manually coupled. The N-Fmoc-protected amino acid (3 equiv.) was preactivated with 1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium (HATU, 3 equiv.) and N,N-diisopropylethylamine (DIPEA, 3 equiv.) in the minimal volume of dimethylformamide (DMF) and was added to the resin. The mixture was allowed to react in an orbital shaker with stirring for 30 min. Then, peptide chains were elongated on a CEM Liberty Blue microwave peptide synthesizer. Fluorenylmethoxycarbonyl protecting group (Fmoc) deprotection was carried out using 10% (w/v) piperazine and 0.1 M OxymaPure in a 9:1 mixture of N-methyl-2-pyrrolidone (NMP) and ethanol (EtOH). The N-Fmoc-protected amino acids (5 equiv., 0.2 M in DMF) were added with OxymaPure (5 equiv., 1 M in DMF) and N,N′-Diisopropylcarbodiimide (DIC, 5 equiv., 0.5 M in DMF) to the resin. The mixtures were stirred for 3 min at 90° C., except for Cys, His and Arg residues, which were coupled at 50° C. for 10 min. The N-terminal Cys residue in SEQ ID NO: 5 was introduced using a Boc-amino acid. After chain elongation, the resin was washed extensively with DMF and of 1,2-dichloroethane. 4-nitrophenyl chloroformate was dissolved in the least amount of 1,2-dichloroethane, added to the resin and left to gently agitate for 1 h at room temperature. The resin was washed with DCM and DMF, and reacted for 30 min with a 0.5 M solution of DIPEA in anhydrous DMF. Finally, peptides were cleaved with concomitant removal of the side-chain protecting groups, using a 92.5:2.5:2.5:2.5 mixture of trifluoroacetic/water/triisopropylsilane/3,6-dioxa-1,8-octanedithiol (TFA/water/TIS/DODT) for 2 h at room temperature. This way the linear peptide precursor with SEQ ID NO: 5 (CVRRPRRRLCFVRPKFVV) was obtained.

The ligation buffer (6M guanidinium hydrochloride, 200 mM sodium phosphate, 20 mM Tris(2-carboxyethyl)phosphine (TCEP), 100 mM 4-mercaptophenol, pH 7) was freshly prepared and bubbled with nitrogen. The peptide of SEQ ID NO: 5 was dissolved at a 2-3 mM concentration and the solution was stirred at rt for 4 h. Then, the reaction was acidified, extracted with tert-butyl methyl ether (TBME 2×50 mL) and loaded on a PoraPakC18 Cartridge for desalting. The guanidinium salts were washed with buffer, while the peptide was eluted at the end in water/acetonitrile (H₂O/ACN (1:1)) and freeze-dried. To yield the final bicyclic peptides of SEQ ID NO: 4, intramolecular disulfide bonds were formed under highly diluted conditions (20-40 μM), by stirring an aqueous solution of the peptides (pH 8) under air oxygen for 24 h. Peptides were purified by semi-preparative HPLC on a Waters 2700 sample manager equipped with a Waters 2487 dual-wavelength absorbance detector, a Waters 600 controller, a Waters fraction collector and Masslynx software by using a Sunfire C18 column (150×10 mm×3.5 μm, 100 Å, Waters), flow rate 6.6 mL/min, solvent A=0.1% trifluoroacetic acid in water; solvent B=0.1% trifluoroacetic acid in acetonitrile.

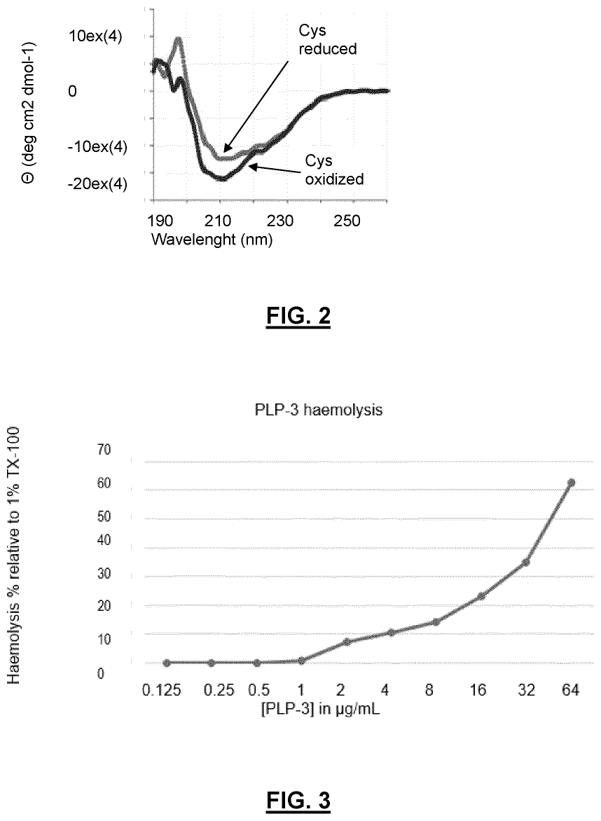

The conformational propensity of the peptide of SEQ ID NO: 4 in solution was analysed by circular dichroism (CD) spectroscopy. The spectrum of PLP-3 at physiological pH and 25° C. showed a minimum at ca. 212 nm, a negative broad band at 220 nm, and a maximum at 201 nm, which altogether represent the typical signature of a β-hairpin-stabilized structure. Reduction of the disulfide bond to yield the monocyclic peptide resulted in a small decrease in CD signal intensity, thus suggesting that although the cyclic peptide is already well folded into an antiparallel β-sheet structure, the disulfide staple present in the bicyclic peptide further contributes to the stability of this conformation. In FIG. 2 the CD spectra of the bicyclic peptide of SEQ ID NO: 4 (named PLP-3) and the corresponding Cys-reduced PLP-3 (cycled SEQ ID NO: 5 with cysteines in reduced form; i.e., alpha carbon with the —SH group) at pH 7.4 and 25° C. are shown.

Circular dichroism spectra were recorded using a Jasco 810 UV-Vis spectropolarimeter, equipped with a CDF 426S/426L peltier. Peptide samples (reduced and oxidised) were dissolved in PBS buffer and spectra were recorded at concentrations of 100 μM. TCEP (5 mM) was added to the reduced peptide sample to prevent disulfide bond formation. Additional readings were done after adding 20% or 40% trifluoroethanol (TFE) to the samples. The following parameters were used: sensitivity (standard, 100 mdeg), start (260 nm), end (190 nm), data pitch, (0.5 nm), scanning mode (continuous), scanning speed (200 nm/min), response (1 s), band width (1.0 nm), and accumulation (3). A blank spectrum of the buffer was subtracted from all recordings, and the resulting spectra were smoothed using the Savitzky-Golay method (convolution width=21) and taken to zero at the far-UV region (A=260 nm).

### Example 2. Antimicrobial (i.e., Antibacterial) Activity Testing of the Peptides

Minimum Inhibitory Concentration (MIC) of a peptide of SEQ D NO: 4 (abbreviated PLP-3) and colistin against a collection of clinically relevant and commercially available bacterial strains was obtained via broth microdilution. The protocol for antimicrobial activity determination, detailed below, consisted in a modification of Clinical & Laboratory Standards Institute (CLSI) Guidelines.

MIC is the value defined as the lowest concentration of an antimicrobial, in these tests either PLP-3 or colistin, which inhibits the macroscopic growth of a bacterial strain after 18-22 h incubation at 37° C. The results from these assays are obtained via visual identification of the bacterial bottom at the end of the wells of the testing plate. Since the wells contain decreasing concentrations of the compounds, the final result is indicated as a concentration, MIC, expressed in μg/mL of the agent (in this case PLP-3 or colistin).

Bacterial strains were streaked in Columbia Agar plates (Columbia Agar with 5% Sheep Blood 90 mm Stacker plates, BD) and incubated overnight at 37° C. Polypropylene round bottomed 96-well plates were filled with 50 μL/well of Mueller-Hinton Broth (MHB, CM0405, 500 g, ThermoFisher Scientific™ Oxoid™) and 50 μL of either colistin or PLP-3 (at 4× the chosen initial concentration of the test) were added to the first well of each row of the plate followed by serial dilutions up until the 10th column of the plate.

Finally, strain inoculums were prepared by diluting 1:100 0.5 McFarland solutions in MHB. Plates were then inoculated with 50 μL of the inoculums and covered with film before 18 to 22 h incubation at 37 C.

Three biological and three technical replicates were performed per strain.

Initial PLP-3 antimicrobial activity screening was performed against a colistin susceptible and colistin resistant strain of each of the following species: A. baumannii, K. pneumoniae and P. aeruginosa (results not included). These assays confirmed potent antimicrobial activity through these species and resulted in testing a larger panel of strains of each species containing multidrug resistant (MDR), colistin susceptible and colistin resistant strains.

Broth microdilution testing of 15 strains of each species provided MIC₅₀ and MIC₅₀ values (Table 4). Strains were either from clinical origin, relevant to current clinical situation, or commercially available strains belonging to the American Type Culture Collection (ATCC), namely: A. baumannii 17978 and 19606, K. pneumoniae 13883 and P. aeruginosa 27853.

This panel represents an example of relevant MDR, colistin resistant and colistin susceptible strains from which to compare results. The minimum inhibitory concentration (MIC) is the lowest concentration of a chemical, usually a drug, which prevents visible growth of a bacterium or bacteria. MIC₅₀ represents the antimicrobial concentration value that inhibits the growth of 50% of the bacterial strains and MIC₅₀ for 90% of each species from the tested panel of strains. Being colistin both a peptide and a last-resource antibiotic, it was included in these assays for comparison against a commercially available antibiotic.

PLP-3 revealed a potent antibiotic activity against the collection of A. baumannii strains. Range of MICs for the collection was the narrowest, from 1 to 2 μg/mL. This translated into an MIC₅₀ value of 1 μg/mL and MIC₅₀ of 2 μg/mL. PLP-3's activity was kept even against colistin resistant A. baumannii strains.

TABLE 1
PLP-3 and colistin MIC values (i.e., the lowest concentration of the
bicyclic peptide, which prevents visible growth of bacteria.)
for a panel of A. baumannii strains.

| | | | MICs | |
|---|---|---|---|---|
| | | | SEQ ID NO: 4 | |
| Species | # | Strain | (PLP-3) | Colistin |
| Acinetobacter | 1 | 8* | 1 | 0.5 |
| baumannii | 2 | 19* | 2 | 0.25 |
| | 3 | 80* | 2 | 0.5 |
| | 4 | 174* | 2 | 0.25 |
| | 5 | 180* | 1 | 0.5 |
| | 6 | 226* | 1 | 0.25 |
| | 7 | 253* | 2 | 0.25 |
| | 8 | 317 | 1 | 0.25 |
| | 9 | 383 | 2 | 0.25 |
| | 10 | 19606 | 2 | 0.5 |
| | 11 | CR17* | 1 | >64 |
| | 12 | CR49 | 1 | 64 |
| | 13 | CR86 | 1 | 64 |
| | 14 | 210* | 2 | 8 |
| | 15 | ATCC 17978 | 1 | 0.25 |
| Pseudomonas | | ATCC 27853 | 2 | ≤0.125 |
| aeruginosa | | | | |

*MDR strains: not susceptible to at least 1 agent in at least 3 different antimicrobial categories used for the effective treatment of infections caused by such pathogen.

For P. aeruginosa strains, PLP-3's MIC range was 1 to 8 μg/mL. Still with an MIC₅₀ value of 4 μg/mL and MIC₅₀ of 8 μg/mL, PLP-3 had antibiotic activity throughout colistin resistant and susceptible strains (see data in Table 2).

Table 2. PLP-3 and colistin MIC values for Pseudomonas aeruginosa strains. * MDR strains: not susceptible to at least 1 agent in at least 3 different antimicrobial categories used for the effective treatment of infections caused by such pathogen.

TABLE 2
PLP-3 and colistin MIC values for Pseudomonas aeruginosa strains.

| | | | MICs | |
|---|---|---|---|---|
| | | | SEQ ID NO: 4 | |
| Species | # | Strain | (PLP-3) | Colistin |
| Pseudomonas | 1 | R1 | 1 | >64 |
| aeruginosa | 2 | R2 | 4 | 64 |
| | 3 | R3* | 4 | 32 |
| | 4 | S1 | 2 | ≤0.125 |
| | 5 | S2* | 2 | ≤0.125 |
| | 6 | S3 | 4 | 0.5 |
| | 7 | S4 | 1 | ≤0.125 |
| | 8 | S5 | 4 | ≤0.125 |
| | 9 | 36a | 2 | ≤0.125 |
| | 10 | 38a | 2 | 0.25 |
| | 11 | ATCC 27853 | 2 | ≤0.125 |
| | 12 | C1 | 8 | 0.5 |
| | 13 | C2 | 8 | 1 |
| | 14 | C3 | 4 | 0.5 |
| | 15 | C4 | 4 | 0.5 |

*MDRstrains: not susceptible to at least 1 agent in at least 3 different antimicrobial categories used for the effective treatment of infections caused by such pathogen.

The widest MIC range was found for the K. pneumoniae collection, from 2 to 32 μg/mL. MCI₅₀ of this collection was 4 μg/mL and MIC₉₀ 16 μg/mL (See Table 3).

TABLE 3
PLP-3 and colistin MIC values for Klebsiella pneumoniae strains.

| | | | MICs | |
|---|---|---|---|---|
| | | | SEQ ID NO: 4 | |
| Microorganism | # | Strain | (PLP-3) | Colistin |
| Klebsiella | 1 | 145* | 8 | 32 |
| pneumoniae | 2 | 398* | 16 | >64 |
| | 3 | 674* | 4 | 0.25 |
| | 4 | 681* | 16 | >64 |
| | 5 | 716* | 4 | 0.5 |
| | 6 | 741* | 4 | 0.5 |
| | 7 | 1052* | 16 | >64 |
| | 8 | 1129* | 4 | 0.5 |
| | 9 | ATCC 13883 | 2 | 1 |
| | 10 | 545 | 2 | 0.25 |
| | 11 | 851* | 2 | 0.5 |
| | 12 | 994* | 32 | >64 |
| | 13 | 1078* | 16 | 64 |
| | 14 | 1478* | 16 | >64 |
| | 15 | 1480* | 2 | 0.25 |
| Pseudomonas | | ATCC 27853 | 2 | 0.25 |
| aeruginosa | | | | |

* MDR strains: not susceptible to at least 1 agent in at least 3 different antimicrobial categories used for the effective treatment of infections caused by such pathogen.

P. aeruginosa ATCC 27853 strain (commercially available from the American Type Collection Culture) was used as control by performing MICs to colistin and PLP-3 in all broth microdilution assays

In the next Table 4, determined MIC₅₀ and MIC₅₀ values are indicated.

TABLE 4
MIC₅₀ and MIC₉₀ values (in μg/mL) for
SEQ ID NO: 4 (PLP-3) and colistin

| | PLP-3 | | Colistin | |
|---|---|---|---|---|
| Species | MIC₅₀ | MIC₉₀ | MIC₅₀ | MIC₉₀ |
| A. baumannii | 1 | 2 | 0,5 | 64 |
| K. pneumoniae | 4 | 16 | 8 | >64 |
| P. aeruginosa | 4 | 8 | 0,5 | 64 |

### Example 3. Albumin MICs of the Peptides

To assess antimicrobial activity of PLP3 in a physiological relevant environment, MICs were performed at physiological human albumin concentrations. To this end, Mueller-Hinton broth (MHB) was supplemented with 40 mg/mL human albumin (A1653-5G, Merck) and broth microdilution protocol was used. SEQ ID NO: 4 (PLP-3) and colistin stocks were prepared both in MHB and albumin supplemented MHB. These were used for comparison of MICs in the presence or absence of albumin, and as a control.

There was a 3-fold difference for P. aeruginosa R2 strain, however no such differences were seen for the rest of the strains tested. It is hypothesized that even if free peptide was scarce due to binding to albumin, A baumannii strains' MICs are low enough that little peptide concentration is effective to inhibit their growth (MIC 90 of 2 μg/mL). Data are listed in table 5.

TABLE 5
MICs in the presence of albumin and without albumin (w/o)

| | | PLP-3 (SEQ ID NO: 4) MIC (in μg/mL) | | |
|---|---|---|---|---|
| | | With Albumin 40 | | |
| Species | Strains | mg/mL | w/o Albumin | Folds |
| A. baumannii | CR17 | 2 | 1 | 1 |
| | ATCC | 4 | 1 | 2 |
| | 17978 | | | |
| P. aeruginosa | R2 | 32 | 4 | 3 |
| | ATCC | 8 | 2 | 2 |
| | 27853 | | | |

### Example 4. Erythrocyte Haemolysis Assay

A 50% haematocrit solution (1:1 PBS 1×, P5493, 1 L, Sigma) was obtained from a commercially available human blood sample by performing 3 consecutive washes with PBS 1× and centrifuging at 2600 rpm for 10 minutes between washes. A final 4% erythrocyte solution was prepared with PBS 1× and chilled on ice until used.

Colistin and SEQ ID NO: 4 (PLP-3) stocks were prepared at 256 μg/mL in MHB and left at 4 C until used.

96-well, round bottom plates were filled with 50 μL PBS 1× but for the haemolysis controls, which were left empty. 50 μL of each antimicrobial were added to the first column of each plate and serial dilution was performed. Then 50 μL of 4% erythrocyte solution were added to all wells of the plate and gently resuspended. Positive control consisted in PBS 1× and erythrocytes while haemolysis control was settled at 1% Triton™ X-100 (TX-100, 9002-93-1, 100 mL, Sigma) and erythrocytes.

Plates were incubated for 4 h at 37° C. and then centrifuged at 1500 rpm 5 min. From each well, 80 μL of the supernatant were transferred to a new 96-well, polystyrene, flat bottom plate by carefully tilting the plate to avoid transferring the well's erythrocyte pellet. An EPOCH™ spectrophotometer was used to read the plates at 450 nm.

Haemolysis assays of human erythrocytes were done using 1% TX-100 as a control for total haemolysis and absorbance values of PLP-3 were represented in relation to total haemolysis (TX-100 values).

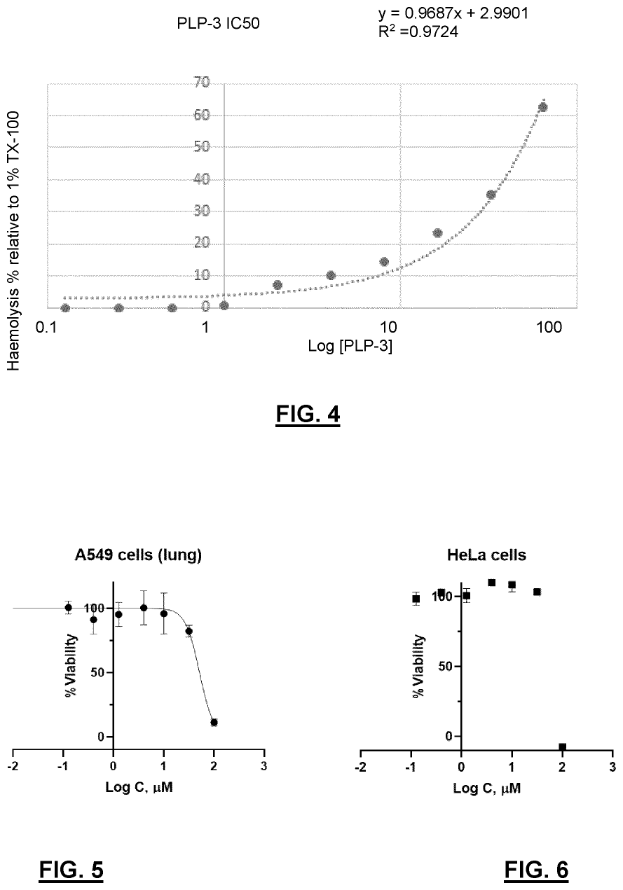

For up to 16 μg/mL (maximum MIC90 value for the three species tested) erythrocyte haemolysis is kept under 25%. Haemolysis up to 2 μg/mL PLP-3 is under 10%, suggesting low toxicity. These data are depicted in FIGS. 3 and 4, respectively.

IC₅₀ value at 48.53 μg/mL was obtained from the haemolysis curve. This value relates to those found in in vitro toxicity assays with A549 and HeLa cells. Data are listed in table 6.

TABLE 6
IC₅₀ values

| PLP-3 | PLP-3 |
|---|---|
| haemolysis % | haemolysis |
| relative to 1% | IC₅₀ (ug/mL) |
| TX-100 at 64 | |
| ug/mL PLP-3 | |
| 62,57 | 48,53 |

### Example 5. Cytotoxicity in Human Cells

In vitro toxicity of PLP-3 was performed via XTT viability assays against HeLa and A549 cells.

Cell viability assay (XTT): HeLa and A549 human cancer cells (both commercially available from different providers) were seeded in a 96-well Corning microplates at two initial densities (2×10³ cells/well and 5×10³ cells/well). After 24 h incubation at 37° C., cells were treated with the peptide dissolved in Dulbecco's Modified Eagle's Medium (high glucose, 10% FBS) for 48 or 72 h (100 μL, concentration range 0.1-100 μM). Then, the peptide solution was removed and substituted by fresh medium. 50 μL of activated-XTT solution (0.1 mL of the activation reagent in 5.0 mL of the XTT reagent, mixed 15 min before use) was added to each well and incubated for 4 h at 37° C. The absorbance at 450 nm was measured in a PowerWave X reader (Bio-Tek, INC), where the positive control was cells treated with DMSO and the negative control was untreated cells. Each experiment was performed in triplicate.

FIG. 5 (A459 human lung cancer cells) and in FIG. 6 (HeLa cells), both represent the viability percentage (% viability) in relation to the logarithm of the concentration (log C (μM)) of the bicyclic peptide. As it can be extracted from these figures, the bicyclic peptides of the invention were not toxic for the cells.

All these data in the examples confirm that the cyclic peptides of the invention (comprising a well-defined β-hairpin conformation of polycationic nature and with amphipathic character and with a tight intramolecular packing), serve the purpose of potent Gram-negative antibacterial compounds. Moreover, the peptides of the invention are highly selective for bacteria as they exhibit very low toxicity for human cells.

### Example 6. Antimicrobial Activity on Gram-Positive Species

The Minimum Inhibitory Concentration (MIC) of PLP-3 peptide (SEQ D NO: 4) against a collection of clinically relevant Gram-positive bacterial strains was obtained via broth microdilution. The tested species Staphylococcus aureus, Enterococcus faecium and Enterococcus faecalis. The followed protocol was based on the Clinical & Laboratory Standards Institute (CLSI) and EUCAST Guidelines.

In these tests, MIC is the value defined as the lowest concentration of an antimicrobial which inhibits the macroscopic growth of a bacterial strain after 16-20 hours of incubation at 37° C. The results of this assay were obtained by visual identification of bacterial growth at the bottom of each well plate. Since the wells contained decreasing concentrations of the peptide, the final result was indicated as a concentration, MIC, expressed in μg/mL of the PLP-3.

Bacterial strains were streaked in Columbia Agar plates (Columbia Agar with 5% Sheep Blood 90 mm Stacker plates, Becton Dickinson) and incubated overnight at 37° C. Polypropylene round bottomed 96-well plates were filled with 50 μL/well of Cation Adjusted Mueller-Hinton Broth (CAMHB, 90922, 500 g, Sigma-Aldrich) and 50 μL of PLP-3 (at 4 times higher the chosen initial concentration of the test) were added to the first well of each row of the plate followed by 1:2 serial dilutions up until the 10^{th} column of the plate. The 11^{th} and 12^{th} columns were used for positive and negative growth controls.

Stain inoculums were standardized by preparing a 1:100 dilution of 0.5 McFarland solutions in CAMHB. Plates were inoculated with 50 μL of the diluted inoculums and covered with an adhesive film before 16-20 hours of incubation at 37° C.

Two biological and two technical replicates were performed per strain.

Broth microdilution testing from a panel of clinical strains of each specie provided MIC₅₀ and MIC₅₀ values (Table 7). Strains were either from clinical origin, relevant to current clinical situation, or commercially available strains belonging to the American Type Culture Collection (ATCC), S. aureus 29213. Regarding clinical strains collection, 10 out of the 19 S. aureus strains were resistant to methicillin (MRSA), and 11 out of the 22 E. faecium strains were resistant to Vancomycin (VRE).

MIC₅₀ value represents the antimicrobial concentration value that inhibits the growth of 50% of the bacterial strain panel. MIC₅₀ value represents the antimicrobial concentration value that inhibits the growth of 90% the bacterial strain collection.

As shown in Table 7, below, PLP-3 exhibited a potent antibiotic activity also against high-priority and clinically relevant Gram-positive bacterial strains.

TABLE 7
PLP-3 MIC values (i.e., the lowest concentration of the
bicyclic peptide, which prevents visible growth of bacteria)
of a panel of different Gram-positive species: S.
aureus (10/19 MRSA), E. faecium (11/22 VRE), E. faecalis.

| | Number of | | |
|---|---|---|---|
| Specie | strains | MIC₅₀ [μg/mL] | MIC₉₀ [μg/mL] |
| Staphylococcus | 19 | 4 | 8 |
| aureus | | | |
| Enterococcus | 22 | 2 | 2 |
| faecium | | | |
| Enterococcus | 15 | 4 | 4 |
| faecalis | | | |

### Example 7. PLP-3 Time-Kill Curves

Time-kill curves (TKC) determine the in vitro antimicrobial activity of a compound, monitoring the bacterial growth over a certain period of time.

This assay was performed for two Acinetobacter baumannii strains (a clinical strain and ATCC 19606 strain) and one Pseudomonas aeruginosa ATCC 27853 strain, monitoring its growth during 24 hours at different PLP-3 (SEQ ID NO: 4) concentrations: MIC, 2×MIC, 4×MIC, 8×MIC. Moreover, in each TKC assay a negative and a positive growth control was stablished.

In different 13MI tubes, a certain concentration of PLP-3 peptide, Mueller-Hinton Broth (MHB, 1214, Condalab) and a fixed inoculum concentration were added. The final inoculum concentration in each tube was 5-105 CFU/MI. Tubes were incubated at 37° C. at 180 rpm during 24 hours. Aliquots of the liquid culture were taken at different times: 0 h, 10 minutes, 30 minutes, 1 h, 2 h, 4 h, 8 h and 24 h. For each taken aliquot, serial dilutions with Phosphate Saline Buffer 1× (PBS 1×) were made and streaked in Luria-Bertani Agar (LB, 1083, Condalab). LB Agar plates were incubated at 37° C. during 18-22 hours. Grown colonies in LB Agar were counted in order to quantify the inoculum in each time period. From colony counting, Colony Forming Units per MI (CFU/MI) were determined, and log(CFU/MI) were represented in a line chart graph along the 24 hours of incubation in order to determine if PLP-3 had a bactericidal or bacteriostatic activity (depending on whether the decrease is greater or less than 3 logarithms, respectively).

Three biological replicates were performed for each strain, and the mean and standard deviation was determined each time.

As shown in Tables 8, 9, and 10 below, the application of PLP-3 on A. baumannii 8 clinical strain, A. baumannii ATCC 19606, or Pseudomonas aeruginosa ATCC 27853, caused a bactericidal effect over long periods of time even at the lowest concentrations tested.

TABLE 8
Log(CFU/mL) Mean and Standard Deviation (SD) of three time-kill curves
replicates of A. baumannii 8 clinical strain.

| | Positive control | | MIC | | 2xMIC | | 4xMIC | | 8xMIC | |
|---|---|---|---|---|---|---|---|---|---|---|
| Time | log(CFU/mL) | | log(CFU/mL) | | log(CFU/mL) | | log(CFU/mL) | | log(CFU/mL) | |
| (hours) | Mean | SD | Mean | SD | Mean | SD | Mean | SD | Mean | SD |
| 0.00 | 5.60 | 0.17 | 5.60 | 0.17 | 5.60 | 0.17 | 5.60 | 0.17 | 5.60 | 0.17 |
| 0.17 | 5.67 | 0.06 | 5.63 | 0.04 | 5.56 | 0.10 | 5.30 | 0.37 | 4.45 | 0.66 |
| 0.50 | 5.69 | 0.03 | 5.18 | 0.06 | 4.92 | 0.09 | 4.11 | 0.24 | 0.00 | 0 |
| 1.00 | 6.02 | 0.22 | 4.55 | 0.48 | 4.20 | 0.49 | 2.74 | 0.87 | 0.00 | 0 |
| 2.00 | 6.40 | 0.42 | 3.56 | 0.44 | 2.74 | 0.39 | 0.00 | 0.00 | 0.00 | 0 |
| 4.00 | 7.56 | 0.20 | 2.54 | 0.45 | 1.89 | 1.66 | 0.00 | 0.00 | 0.00 | 0 |
| 8.00 | 8.48 | 0.36 | 2.90 | 0.32 | 1.05 | 1.83 | 0.00 | 0.00 | 0.00 | 0 |
| 24.00 | 8.87 | 0.29 | 4.67 | 0.83 | 1.49 | 2.58 | 0.00 | 0.00 | 0.00 | 0 |

MIC, Minimum Inhibitory Concentration; SD, Standard Deviation.
MIC (2 μg/mL), 2xMIC (4 μg/mL), 4xMIC (8 μg/mL), 8MIC (16 μg/mL).

TABLE 9
Log(CFU/mL) Mean and Standard Deviation (SD) of three time-kill curves
replicates of A. baumannii ATCC 19606.

| | Positive control | | MIC | | 2xMIC | | 4xMIC | | 8xMIC | |
|---|---|---|---|---|---|---|---|---|---|---|
| Time | log(CFU/mL) | | log(CFU/mL) | | log(CFU/mL) | | log(CFU/mL) | | log(CFU/mL) | |
| (hours) | Mean | SD | Mean | SD | Mean | SD | Mean | SD | Mean | SD |
| 0.00 | 5.87 | 0.06 | 5.87 | 0.06 | 5.87 | 0.06 | 5.87 | 0.06 | 5.87 | 0.06 |
| 0.17 | 5.98 | 0.04 | 5.85 | 0.18 | 5.54 | 0.36 | 5.12 | 0.70 | 3.41 | 0.81 |
| 0.50 | 5.97 | 0.01 | 5.43 | 0.45 | 4.91 | 0.86 | 3.79 | 1.18 | 0.87 | 1.50 |
| 1.00 | 5.93 | 0.08 | 4.01 | 0.98 | 3.14 | 0.85 | 0.94 | 1.62 | 0.00 | 0.00 |
| 2.00 | 6.56 | 0.17 | 2.39 | 0.65 | 2.18 | 0.31 | 0.00 | 0.00 | 0.00 | 0.00 |
| 4.00 | 7.87 | 0.14 | 3.05 | 0.22 | 2.58 | 0.76 | 0.00 | 0.00 | 0.00 | 0.00 |
| 8.00 | 8.15 | 0.18 | 3.85 | 0.69 | 2.56 | 2.22 | 0.00 | 0.00 | 0.00 | 0.00 |
| 24.00 | 8.58 | 0.20 | 5.17 | 0.35 | 3.30 | 2.86 | 0.00 | 0.00 | 0.00 | 0.00 |

MIC, Minimum Inhibitory Concentration; SD, Standard Deviation.
MIC (2 μg/mL), 2xMIC (4 μg/mL), 4xMIC (8 μg/mL), 8MIC (16 μg/mL).

TABLE 10
Log(CFU/mL) Mean and Standard Deviation (SD) of three time-kill curves
replicates of P. aeruginosa ATCC 27853.

| | Positive control | | MIC | | 2xMIC | | 4xMIC | | 8xMIC | |
|---|---|---|---|---|---|---|---|---|---|---|
| Time | log(CFU/mL) | | log(CFU/mL) | | log(CFU/mL) | | log(CFU/mL) | | log(CFU/mL) | |
| (hours) | Mean | SD | Mean | SD | Mean | SD | Mean | SD | Mean | SD |
| 0.00 | 5.52 | 0.16 | 5.52 | 0.16 | 5.52 | 0.16 | 5.52 | 0.16 | 5.52 | 0.16 |
| 0.17 | 5.41 | 0.16 | 4.81 | 0.34 | 4.48 | 0.09 | 4.32 | 0.54 | 4.14 | 0.68 |
| 0.50 | 5.47 | 0.02 | 4.34 | 0.39 | 3.75 | 0.42 | 3.46 | 0.32 | 1.43 | 1.37 |
| 1.00 | 5.41 | 0.03 | 3.67 | 0.08 | 2.97 | 0.84 | 1.23 | 1.08 | 0.00 | 0.00 |
| 2.00 | 6.18 | 0.46 | 3.15 | 0.22 | 0.88 | 1.53 | 0.57 | 0.98 | 0.00 | 0.00 |
| 4.00 | 7.43 | 0.32 | 2.63 | 0.41 | 0.00 | 0.00 | 0.00 | 0.00 | 0.00 | 0.00 |
| 8.00 | 8.72 | 0.61 | 2.10 | 0.17 | 0.00 | 0.00 | 0.00 | 0.00 | 0.00 | 0.00 |
| 24.00 | 8.90 | 0.03 | 5.08 | 1.78 | 0.00 | 0.00 | 0.00 | 0.00 | 0.00 | 0.00 |

MIC, Minimum Inhibitory Concentration; SD, Standard Deviation.
MIC (8 μg/mL), 2xMIC (16 μg/mL), 4xMIC (32 μg/mL), 8MIC (64 μg/mL).

### CITATION LIST

### Patent Literature

EP1363934B11
EP1532164B11
EP1622931B1
EP2978771B1
EP2978772B1

### Non Patent Literature

Jang, H. et al., “Conformational Study of the Protegrin-1 (PG-1) Dimer Interaction with Lipid Bilayers and Its Effect”, BMC Struct. Biol.—2007, vol. no. 7
Uper et al, in “Emerging peptide antibiotics with therapeutic potential”, Medicine in Drug Discovery 9, (2021) 100078
Shankaramma et al., “Macrocyclic Hairpin Mimetics of the Cationic Antimicrobial Peptide Protegrin I: A new Family of Broad-Spectrum Antibiotics”, ChemBionChem—2002, vol. no. 3, pp.: 1126-1133
Tam et al. in “Membranolytic selectivity of cystine-stabilized cyclic protegrins”, Eur. J. Biochem.—2000, vol. no. 267, pp.: 3289-3300
Robinson et al. (2005), “Properties and structure-activity studies of cyclic β-hairpin peptidomimetics based on the cationic antimicrobial peptide protegrin I” Bioorganic & Medicinal Chemistry 13, 2055-2064
Frecer et al. (2006), “QSAR analysis of antimicrobial and haemolytic effects of cyclic cationic antimicrobial peptides derived from protegrin-1”, Bioorganic & Medicinal Chemistry 14, 6065-6074

What is claimed is:

1. A cyclic peptide of amino acid sequence (1) or a pharmaceutically acceptable salt thereof,

| | (I) |
|---|---|
| (SEQ ID NO: 1) | |
| | X1X2X3X4CX6X7X8PX10X11X12X13CX15X16X17P |

wherein:
each of X1, X2, X3, X8, X10, X16 and X17 are independently a basic amino acid selected from arginine (R), lysine (K), and histidine (H);
each of X4, X7, X12, X13 and X15 are independently a hydrophobic amino acid selected from valine (V), leucine (L), isoleucine (1), and methionine (M);
each of X6, and X11 are independently hydrophobic aromatic amino acids selected from phenylalanine (F), tyrosine (Y) and tryptophan (W);
Proline amino acids at position 9 and 18 are D-proline (D-Pro); and
X1 and D-Pro at position 18 are linked by a peptide bond.

2. The cyclic peptide according to claim 1, which is a bicyclic peptide of amino acid sequence (II), or a pharmaceutically acceptable salt thereof,

| | (II) |
|---|---|
| (SEQ ID NO: 2) | |
| | X1X2X3X4CX6X7X8PX10X11X12X13CX15X16X17P, |

wherein
each of X1, X2, X3, X8, X10, X16 and X17 are independently a basic amino acid selected from arginine (R), lysine (K), and histidine (H);
each of X4, X7, X12, X13 and X15 are independently a hydrophobic amino acid selected from valine (V), leucine (L), isoleucine (1), and methionine (M);
each of X6, and X11 are independently hydrophobic aromatic amino acids selected from phenylalanine (F), tyrosine (Y) and tryptophan (W),
Proline amino acids at position 9 and 18 are D-proline (D-Pro);
Cysteine (C) at position 5 and 14 are linked by a disulfide bond; and
X1 and D-Pro at position 18 are linked by a peptide bond.

3. The cyclic peptide according to claim 1, wherein at least three of the basic amino acids X1, X2, X3, X8, X10, X16 and X17 are arginine (R).

4. The cyclic peptide according to claim 1, wherein all the basic amino acids X1, X2, X3, X8, X10, X16 and X17 are arginine (R).

5. The cyclic peptide according to claim 1, wherein at least one of X6, and X11 is phenylalanine (F).

6. The cyclic peptide according to claim 5, wherein both of X6, and X11 are phenylalanine (F).

7. The cyclic peptide according to claim 1, wherein at least one of X4, X7, X12, X13 and X15 are independently a hydrophobic amino acid selected from valine (V) and leucine (L).

8. The cyclic peptide according to claim 7, wherein X4 is leucine (L) and X12, X13 and X15 are valine (V).

9. The cyclic peptide according to claim 2, with the amino acid sequence cyclo[RRRLCFVRPKFVVCVRRP](SEQ ID NO: 4), in which cysteines at position 5 and 14 are linked by a disulfide bond.

10. A pharmaceutical composition comprising a therapeutically effective amount of the cyclic peptide or a pharmaceutically acceptable salt thereof as defined in claim 1, together with pharmaceutically acceptable excipients and/or carriers.

11.-15. (canceled)

16. A non-therapeutic method for disinfecting or preserving, the method comprising contacting the cyclic peptide or a salt thereof as defined in claim 1 with foodstuffs, cosmetics, nutrient-containing materials, or antiseptic compositions.

17. The cyclic peptide according to claim 1, wherein at least three of the basic amino acids X1, X2, X3, X8, X10, X16 and X17 are arginine (R); at least one of X6, and X11 is phenylalanine (F); and at least one of X4, X7, X12, X13 and X15 are independently a hydrophobic amino acid selected from valine (V) and leucine (L).

18. The cyclic peptide according to claim 1, wherein all the basic amino acids X1, X2, X3, X8, X10, X16 and X17 are arginine (R); both of X6, and X11 are phenylalanine (F); and X4 is leucine (L) and X12, X13 and X15 are valine (V).

19. The method according to claim 13, wherein the gram-negative bacteria is selected from the group consisting of Pseudomonas aeruginosa, Acinetobacter baumannii, and Klebsiella pneumoniae.

20. The method according to claim 14, wherein the gram-positive bacteria is selected from the group consisting of Staphylococcus aureus, Enterococcus faecium, and Enterococcus faecalis.

21. A method for the prevention and/or treatment of a disease caused by a pathogen, the method comprising administering a therapeutically effective amount of a cyclic peptide or a pharmaceutically acceptable salt thereof as defined in claim 1 to a subject in need thereof.

22. The method as defined in claim 21, wherein the pathogen is a gram-positive bacteria.

23. The method as defined in claim 21, wherein the pathogen is a gram-negative bacteria.

24. The method as defined in claim 21, wherein the cyclic peptide or the pharmaceutically acceptable salt thereof is administered in combination with another active agent.

25. A method for the prevention and/or treatment of a disease caused by a pathogen, the method comprising administering a therapeutically effective amount of a pharmaceutical composition as defined in claim 10 to a subject in need thereof.
